(19) United States
(12) Patent Application Publication
(10) Pub. No.: US 2024/0390704 A1
(43) Pub. Date: Nov. 28, 2024

(54) A DEVICE FOR AFFECTING VASCULAR BLOOD FLOW AND METHODS THEREOF

(71) Applicant: NOVAPULSE LTD, Or Yehuda (IL)

(72) Inventors: Shaul Ozeri, Tel-Aviv (IL); Gal Atarot, Kfar Saba (IL); Shai Policker, Raanana (IL)

(21) Appl. No.: 18/714,529

(22) PCT Filed: Nov. 29, 2022
(86) PCT No.: PCT/IL2022/051266

§ 371 Date: May 29, 2024

Related U.S. Application Data
(60) Provisional application No. 63/284,710, filed on Dec. 1, 2021.
(60) Provisional application No. 63/319,392, filed on Mar. 14, 2022.

(51) Int. Cl. A61N 7/00

(52) U.S. Cl. CPC A61N 7/00; A61N 2007/0004; A61N 2007/0047; A61N 2007/0073; A61N 2007/0078

(57) ABSTRACT

The present disclosure relates to an implantable device that delivers therapeutic energy to a patient. More specifically it relates to implantable ultrasonic devices adapted to affect vascular endothelium to induce at least one substance selected from Nitric Oxide, NO, and/or adenosine triphosphate (ATP) release. Furthermore, the present disclosure relates to the treatment of various medical conditions, such as pulmonary hypertension, peripheral arterial disease (PAD) and Asthma by enhancing local perfusion to specific target organs.

### FIELD OF THE INVENTION

The present invention relates generally to an implantable device that delivers therapeutic energy to a patient. More specifically it relates to implantable ultrasonic devices adapted to affect vascular endothelium to induce at least one substance selected from Nitric Oxide, NO, and/or adenosine triphosphate (ATP) release. Furthermore, the present disclosure relates generally to the treatment of various medical conditions, such as pulmonary hypertension, peripheral arterial disease (PAD) and Asthma by enhancing local perfusion to specific target organs.

### BACKGROUND OF THE INVENTION

The emergence of nitric oxide (NO), a reactive, inorganic radical gas as a molecule contributing to important physiological and pathological processes is one of the major biological revelations of recent times.

This molecule is produced under a variety of physiological and pathological conditions by cells mediating vital biological functions. Examples include endothelial cells lining the blood vessels; nitric oxide derived from these cells relaxes smooth muscle and regulates blood pressure and has significant effects on the function of circulating blood cells such as platelets and neutrophils as well as on smooth muscle, both of the blood vessels and also of other organs such as the airways. In the brain and elsewhere nitric oxide serves as a neurotransmitter in non-adrenergic non-cholinergic neurons. In these instances nitric oxide appears to be produced in small amounts on an intermittent basis in response to various endogenous molecular signals. In the immune system nitric oxide can be synthesized in much larger amounts on a protracted basis. Its production is induced by exogenous or endogenous inflammatory stimuli, notably endotoxin and cytokines elaborated by cells of the host defense system in response to infectious and inflammatory stimuli. This induced production results in prolonged nitric oxide release which contributes both to host defense processes such as the killing of bacteria and viruses as well as pathology associated with acute and chronic inflammation in a wide variety of diseases (Furchgott and Zawadzki 1980; Palmer et al. 1987).

In the field of vascular function, it has been reported that NO generation as well as release ATP from endothelial cells (see Matthew A. Muller et al., Augmentation of Tissue Perfusion with Contrast Ultrasound: Influence of Three-Dimensional Beam Geometry and Conducted Vasodilation, Journal of the American Society of Echocardiography, doi:10.1016/j.echo.2021.02.018) is stimulated physiologically when the endothelium is exposed to shear stress induced by blood flow and changes in the blood flow (Taso et al. 1995; Uematsu et al. 1995; Ayajiki et al. 1996; Corson et al. 1996; Fleming et al. 1998). There have been two possible mechanisms of endothelial NO generation in response to fluid shear stress. Previous investigations (Ando et al. 1988; Geiger et al. 1992; James et al. 1995) have established that the exposure of endothelium to increased fluid shear stress may stimulate the release of Ca²⁺ from intracellular stores, with consequent elevation of intracellular free Ca²⁺ concentration, which increases the enzymatic activity of endothelial NO synthase (eNOS) to generate NO.

Recently, however, several investigations (Ayajiki et al. 1996; Corson et al. 1996; Fleming et al. 1998) have reported Ca²⁺ independent activation of the eNOS in response to fluid shear stress. They have suggested that tyrosine phosphorylation and intracellular pH (Ayajiki et al. 1996), phosphorylation of the eNOS (Corson et al. 1996) and tyrosine kinase inhibitor-sensitive pathway (Fleming et al. 1998) play an important role in NO generation.

In addition to this physiological stimulation, recent reports have pointed out that ultrasonic application mimics the required shear force to induced vasodilatation, blood flow increases and pH changes in a frequency and amplitude dependent manner, in light of NO generation from the blood vessels (Mason et al., Augmentation of Tissue Perfusion in Patients With Peripheral Artery Disease Using Microbubble Cavitation, JACC Cardiovasc Imaging 2020 March;13(3):641-651; Belcik et al., Augmentation of limb perfusion and reversal of tissue ischemia produced by ultrasound-mediated microbubble cavitation, Circ. Cardiovasc Imaging. 2015 April;8(4):e002979; Kiyoshi Iida et al. Noninvasive low-frequency ultrasound energy causes vasodilation in humans; J Am Coll Cardiol. 2006 Aug. 1;48(3):532-7; Muller et al., Treatment of Limb Ischemia with Conducted Effects of Catheter-Based Endovascular Ultrasound, Ultrasound Med Biol. 2021 August;47(8):2277-2285;).

One of the most prominent possible use of NO induction as well as release of adenosine triphosphate is in the treatment of peripheral arterial disease (PAD) ischemic tissue. PAD is the narrowing or blockage arteries by atherosclerosis (the buildup of fatty plaque) with or without calcifications.

Narrowing or PAD can happen in any blood vessel, but it is more common in the legs or lower extremities. PAD is a chronic disease. At its moderate form it is presented as claudication. At its severe form, PAD is presented as Critical limb ischemia (CLI). CLI is a severe blockage in the arteries of the lower extremities, which markedly reduces blood-flow. Peripheral arterial disease (PAD) and ischemic tissue is the most common form of atherosclerosis that affects many people worldwide. As a result of such disease, patients' mobility is limited (claudication) and their quality of life is significantly impacted. Such disease can progress to more severe stages and eventually present a risk of amputation requiring revascularization treatment (e.g. Artery bypass, stenting or angioplasty, opening the arteries in a cathlab and placing a stent), but in the earlier stages, many people experience pain during physical activity (e.g., walking); in severe cases, patients will feel pain in the feet or toes, even while resting. Complications of poor circulation can include sores and wounds that won't heal in the legs and feet. Left untreated, the complications of CLI may result in amputation of the affected limb. Such condition may be treated medically with exercise and drugs, such as Cilostazol, which modestly improves walking ability by inhibition of platelet aggregation. However, in many cases, patients do not follow the prescribed exercise therapy because of pain associated with the disease.

Medical interventions such as balloon angioplasty, stenting, and surgery are options to treat patients who are suffering from peripheral arterial diseases and critical limb ischemia. However, many of such procedures may fail. Consequences of graft failure include continued ischemia, poor wound healing, gangrene, or amputation of a patient's limb.

The vasodilative effect of Nitric Oxide (NO) and adenosine triphosphate (ATP), its positive effect on perfusion and the release of NO from the endothelia (internal layer) of blood vessels is a major natural mechanism to control local and systemic blood pressure. It is also known that administration of external NO can induce local vasodilation, but the therapeutic use of systemic NO is limited because of its short half-life (less than 1 sec) and the potential harmful effects that high doses could have on systemic blood pressure.

Another use of NO and ATP induction is for primary pulmonary hypertension. Pulmonary hypertension (PH) or Pulmonary arterial hypertension (PAH) is an increase in blood pressure in the pulmonary artery and/or capillaries, together known as the lung vasculature.

PH is a disease phenomenon of multifactorial etiology with high mortality. The disease causes increased work for the right side of the heart and eventually hypertrophy and dysfunction, often in both the right and left side of the heart. Despite improvements in therapy, the prognosis of pulmonary hypertension is poor, with median survival being around 5 years.

Typically, patients suffering from PH are treated with pharmacological agents which are extremely expensive and not entirely-efficacious. Moreover, treatment outcome amongst patients suffering from PH is highly variable, mainly due to the variance in underlying factors.

Thus, the present invention also relates to improving NO release in ischemic tissue by applying ultrasound to the tissue under conditions effective to increase blood flow to said ischemic tissue (e.g., treating ischemic limbs or tissue affected by peripheral arterial disease). In other words, as nitric oxide has been proven a potent vasodilator with great therapeutic potential, there is still a long felt need to locally increase availability of nitric oxide at the target tissue (by utilization of ultrasonic energy) to treat the above disclosed tissues.

Furthermore, it was found that the application of Ultrasound energy to increase NO and ATP release by an implantable and/or wearable devices requires significant energy focused on the blood vessel; yet more, the continuous application of such energy might result in a reduced efficacy by mechanism of physiological adaptation or other. Thus, to maintain high efficacy, it is beneficial to minimize the application of energy to periods that coincide with natural physiological demand and to limit the duration of such stimulation to an optimum level after which the marginal increase in NO production and vasodilation is diminished.

Several devices are known in the art. One of which is described in PCT publication no. WO2018071908 to VIBRATO MEDICAL INC, which discloses wearable, non-invasive ultrasound modalities for treating a variety of medical conditions, including but not limited to peripheral vascular disease. The modality could be therapeutic ultrasound (TUS), and be configured to promote angiogenesis within a patient via stimulation of cavitation and shear stress, among other mechanisms. However, said device is wearable and its effect is limited (in depth of penetration). It should be further noted that such a device is a bulky device (adapted to be wearable), and requires the presence of an acoustic gel between the transducers and the skin surface. Therefore, the acceptance rate of the patients is low and as such (low acceptance rate and gel requirements) it does not suit for long term use.

It is therefore an object of this invention to use sensor embedded within the implantable device, external to the body (e.g., a wearable sensor) and/or implantable elsewhere in the body to detect the periods in which the patient physiology creates an increased demand for oxygenated blood and enhanced perfusion and thereafter apply energy (e.g., Ultrasound) to initiate stimulation of NO and ATP production during that time. It is also the object of this invention to test the period which is optimal for NO and ATP production and limit the time of stimulation to these periods.

### SUMMARY OF THE INVENTION

The above disclosed long felt need can be addressed by providing at least one implantable device that delivers therapeutic energy to a patient. More specifically relates to implantable ultrasonic devices adapted to affect vascular endothelium to induce Nitric Oxide, NO, and ATP release having the features of the independent claim(s). Further features of advantageous embodiments of the present disclosure are the subject matter of the dependent claims.

It is one object of the present invention to provide a device adapted to be implanted adjacent to at least one vessel or tissue containing flowing blood, comprising at least one ultrasonic transducer configured to provide ultrasonic energy to said at least one vessel or tissue containing flowing blood, whereby said ultrasonic energy, when applied, is adapted to cause a physiologic effect in said at least one vessel or tissue; said device is in communication with at least one on-skin remote controller, positioned externally to the patient, adapted to control said ultrasonic energy;
wherein said device is in communication with at least one sensor adapted to monitor at least one physiological state of the patient.

It is another object of the present invention to provide the device as defined above, wherein, upon change of said at least one physiological state, said device is self-activated.

It is another object of the present invention to provide the device as defined above, wherein, upon change of said at least one physiological state, said patient activates said ultrasonic energy or amends said at least one treatment parameter.

It is another object of the present invention to provide the device as defined above, adapted for treatment of pulmonary artery denervation, pulmonary hypertension, ischemic tissues, PAD, CLI, pulmonary artery hypertension, severe asthma patients, improve blood flow to the brain during stroke, increase blood flow to the penis to maintain an erection, enhancement of bioavailability of medications, enhancement of local chemotherapy absorption into a solid tumor by enhancing flow of specific arteries feeding the tumor and any combination thereof.

It is another object of the present invention to provide the device as defined above, wherein said sensor is selected from a group consisting of accelerometer, impedance measurement, Photoplethysmography, PPG sensor, pH sensor, ultrasound sensor, echocardiogram, ultrasound echo, hydrophone, temperature meter, body core temperature, heart pulse rate, Pulse wave properties, glucose sensor,, any sensor indicating a change in cardiac output, any sensor indicating blood pressure, any sensor indicating initiation of a dialysis session, acoustic blood pulse wave sensor, electrocardiogram sensor, ultrasonic sensor, or a vibration sensor, any sensor associated with dialysis machine and any combination thereof; further wherein said sensor is adapted to sense at least one parameter selected from a group consisting of movement of said patient, impedance, PPG signal, pH, acoustic signal, pressure, temperature, heart rate, pulse wave properties, glucose level, blood pressure, and any combination thereof.

It is another object of the present invention to provide the device as defined above, wherein said ultrasonic transducer is an array of transducers.

It is another object of the present invention to provide the device as defined above, wherein the position and orientation of each of said transducers is controllable.

It is another object of the present invention to provide the device as defined above, wherein said array of ultrasonic transducers facilitates a focused ultrasound beam being directed to said at least one vessel or tissue containing flowing blood.

It is another object of the present invention to provide the device as defined above, wherein said array of ultrasonic transducers facilitates beam steering of said ultrasound energy.

It is another object of the present invention to provide the device as defined above, wherein said array of ultrasonic transducers facilitates alignment of ultrasound energy on said at least one vessel or tissue containing flowing blood.

It is another object of the present invention to provide the device as defined above, wherein said array is generating said ultrasonic energy of at least one ultrasonic carrier frequency.

It is another object of the present invention to provide the device as defined above, wherein at least one selected from a group consisting of said at least one on-skin remote controller, said device and any combination thereof comprises at least one coil for forming an inductive link and transcutaneous transfer said ultrasonic energy from said at least one on-skin remote controller to said at least one ultrasonic transducer.

It is another object of the present invention to provide the device as defined above, wherein said at least one on-skin remote controller is adapted to drive said device by at least one method selected from a group consisting of electromagnetic, ultrasonic, capacitive, induction and any combination thereof.

It is another object of the present invention to provide the device as defined above, wherein said at least one on-skin remote controller is adapted to collect data from said at least one sensor and perform at least one selected from a group consisting of: (a) monitor said data; (b) adjust the treatment provided to said patient; (c) maintain the treatment provided to said patient as is; and any combination thereof.

It is another object of the present invention to provide the device as defined above, wherein said data is the shape of the pulse wave signal.

It is another object of the present invention to provide the device as defined above, wherein said data is the time delay between the ECG signal and the pulse wave signal.

It is another object of the present invention to provide the device as defined above, wherein said device is encapsulated in at least one layer of polyetheretherketone, PEEK, wherein the radiating surface of the PEEK is an acoustic matching layer; further wherein the radiating surface of the PEEK is of a quarter lambda, where lambda is the ultrasonic energy wavelength in the PEEK.

It is another object of the present invention to provide the device as defined above, wherein said implant envelope is coated with Parylene.

It is another object of the present invention to provide the device as defined above, wherein said at least one transducer is a piezoelectric transducer made of at least one material selected from a group consisting of lead zirconate titanate, lead magnesium niobate-lead titanate, Hard PZT, composite, PZT with non-uniform polarization, PZT with uniform polarization and any combination thereof.

It is another object of the present invention to provide the device as defined above, wherein said ultrasonic energy may be provided in a continuous manner, or alternatively, in pulses.

It is another object of the present invention to provide the device as defined above, wherein said at least one on-skin remote controller is adapted to measure heart rate from said at least two pulse waves pulses.

It is another object of the present invention to provide the device as defined above, wherein analysis of said at least two pulse wave characteristics facilitate alignment of the position of said implant relatively to said at least one vessel or tissue containing flowing blood.

It is another object of the present invention to provide the device as defined above, wherein at least one of the following is held true (a) said ultrasonic frequency is within 10 kHz-10 MHz; (b) said ultrasonic energy has a peak negative pressure of between about 0.1 MPa and about 2 MPa; and, any combination thereof.

It is another object of the present invention to provide the device as defined above, wherein said ultrasonic energy is synchronized to the pulse wave signal

It is another object of the present invention to provide the device as defined above, wherein said physiological state or change thereof is selected from a group consisting of position of the patient, engagement in physical activity, decrease in NO levels in said at least one vessel or tissue, tissue perfusion, initiation of a physical activity, change in at least one parameter associated with said physical activity, the position of the patient relative to the ground, changes in said position of the patient relative to the ground, application of at least one medical treatment to said patient, changes in application of at least one medical treatment to said patient and any combination thereof.

It is another object of the present invention to provide the device as defined above, wherein, upon change of said at least one physiological state, at least one notification is being sent to said patient or any care giver thereof.

It is another object of the present invention to provide the device as defined above, wherein said physiologic effect is selected from the group consisting of: vasodilation, an increase in local ATP, enhance ATP release, an increase in local nitric oxide, enhance nitric oxide release from the vascular endothelium; prolong local nitric oxide effects; enhance nitric oxide release from red blood cells; an alteration in the function of erythrocytes; a modification in oxygen release from hemoglobin, blood temperature increase; vasodilation; prolong local nitric oxide effects; alteration in the function of erythrocytes; modification in oxygen release from hemoglobin; modification in pH of blood; modulation in the immune response of blood leucocytes; modulation of the coagulation and/or thrombocyte function; modification in the function of heme catalyst enzymes in the blood, improved bioavailability of medication, improved efficiency of a hemodialysis session artery dilation, increased blood perfusion and combinations thereof.

It is another object of the present invention to provide the device as defined above, wherein at least one of said at least one on-skin remote controller is a wearable by said patient; further wherein said at least one on-skin remote controller being a wearable by said patient is selected from a group consisting of integrated in a wearable sock, shoe, glove, clothes, hats and any combination thereof.

It is another object of the present invention to provide the device as defined above, wherein said ischemic tissue is selected from a group consisting of the upper limbs and lower limbs, arms, legs and any combination thereof.

It is another object of the present invention to provide the device as defined above, wherein said at least one on-skin remote controller, adapted to control at least one parameter selected from a group consisting of a phase, an operating frequency, power, intensity, operating amplitude, timing, duration, and any combination thereof of said ultrasonic energy.

It is another object of the present invention to provide the device as defined above, wherein said at least one on-skin remote controller is in communication with said at least one sensor.

It is another object of the present invention to provide the device as defined above, wherein upon change of said at least one physiological state of the patient, at least one of the following is being performed (a) said device is activated and ultrasonic energy is delivered to said at least one vessel or tissue, such that an on-demand treatment is provided; (b) at least one treatment parameter of said ultrasonic energy to said at least one vessel or tissue is amended, such that an as-needed treatment is provided; (c) said change is notified; (d) any combination thereof.

It is another object of the present invention to provide the device as defined above, wherein said sensor is selected from a group consisting of a sensor implanted in said patient, integrated within said device, a sensor being worn by said patient, a remote sensor outside the patient's body, integrated within said at least one on-skin remote controller, and any combination thereof.

It is another object of the present invention to provide the device as defined above, wherein said at least one ultrasonic transducer functions as acoustic sensor and is adapted to sense at least one acoustic wave generated by said at least one vessel or tissue containing flowing blood.

It is another object of the present invention to provide the device as defined above, wherein said device is subcutaneously implanted.

It is another object of the present invention to provide a device adapted to be implanted adjacent to at least one vessel or tissue containing flowing blood, comprising at least one pulse wave sensor, adapted to sense at least one acoustic wave generated by said at least one vessel or tissue containing flowing blood and thereby monitor at least one physiological state of the patient.

It is another object of the present invention to provide the device as defined above, wherein said at least one pulse wave sensor is at least one transducer; further wherein said transducer is selected from a group consisting of capacitive element, accelerometer, piezoelectric element, electromagnetic-acoustic element, electrostatic element and any combination thereof.

It is another object of the present invention to provide the device as defined above, wherein said device is either in communication or additionally comprising at least one sensor selected from a group consisting of accelerometer, impedance measurement, Photoplethysmography, PPG sensor, pH sensor, ultrasound sensor, echocardiogram, ultrasound echo, hydrophone, temperature meter, body core temperature, heart pulse rate, Pulse wave properties, glucose sensor, any sensor indicating a change in cardiac output, any sensor indicating blood pressure, any sensor indicating initiation of a dialysis session, electrocardiogram sensor, ultrasonic sensor, or a vibration sensor, any sensor associated with dialysis machine and any combination thereof; further wherein said sensor is adapted to sense at least one parameter selected from a group consisting of movement of said patient, impedance, PPG signal, pH, acoustic signal, pressure, temperature, heart rate, pulse wave properties, glucose level, blood pressure, and any combination thereof.

It is another object of the present invention to provide the device as defined above, wherein said ultrasonic transducer is an array of transducers.

It is another object of the present invention to provide the device as defined above, wherein said device is encapsulated in at least one layer of polyetheretherketone, PEEK, wherein the radiating surface of the PEEK is an acoustic matching layer.

It is another object of the present invention to provide the device as defined above, wherein said implant envelope is coated with Parylene.

It is another object of the present invention to provide the device as defined above, wherein said at least one transducer is a piezoelectric transducer made of at least one material selected from a group consisting of lead zirconate titanate, lead magnesium niobate-lead titanate, Hard PZT, composite, PZT with non-uniform polarization, PZT with uniform polarization and any combination thereof.

It is another object of the present invention to provide the device as defined above, wherein said physiological state or change thereof is selected from a group consisting of position of the patient, engagement in physical activity, decrease in NO levels in said at least one vessel or tissue, tissue perfusion, initiation of a physical activity, change in at least one parameter associated with said physical activity, the position of the patient relative to the ground, changes in said position of the patient relative to the ground, application of at least one medical treatment to said patient, changes in application of at least one medical treatment to said patient and any combination thereof.

It is another object of the present invention to provide the device as defined above, wherein, upon change of said at least one physiological state, at least one notification is being sent to said patient or any care giver thereof.

It is another object of the present invention to provide the device as defined above, wherein said device is subcutaneously implanted.

It is another object of the present invention to provide an implant being adjacent to, or within at least one vessel or tissue containing flowing blood, comprising:
i. at least one casing comprising at least one at least one coil for forming an inductive link and transcutaneous transfer said ultrasonic energy from at least one on-skin remote controller to at least one ultrasonic transducer;
ii. at least one sealed pig tail wired or wirelessly connected to said at least one casing, comprising said at least one an ultrasonic transducer configured to couple ultrasonic energy to said at least one vessel or tissue containing flowing blood, whereby said ultrasonic energy, when applied, is adapted to cause a physiologic effect in said at least one vessel or tissue;
said casing is in communication with said at least one on-skin remote controller, positioned externally to the patient, adapted to control said ultrasonic energy.

It is another object of the present invention to provide the implant as defined above, wherein at least one selected from a group consisting of said at least one casing, said at least one sealed pig tail and any combination thereof is in communication with at least one sensor adapted to monitor at least one physiological state of the patient.

It is another object of the present invention to provide the implant as defined above, wherein, upon change of said at least one physiological state, said implant is self-activated.

It is another object of the present invention to provide the implant as defined above, wherein, upon change of said at least one physiological state, said patient activates said ultrasonic energy or amends said at least one treatment parameter.

It is another object of the present invention to provide the implant as defined above, adapted for treatment of pulmonary artery denervation, pulmonary hypertension, ischemic tissues, PAD, CLI, pulmonary artery hypertension, severe asthma patients, improve blood flow to the brain during stroke, increase blood flow to the penis to maintain an erection, enhancement of bioavailability of medications, enhancement of local chemotherapy absorption into a solid tumor by enhancing flow of specific arteries feeding the tumor and any combination thereof.

It is another object of the present invention to provide the implant as defined above, wherein said sensor is selected from a group consisting of accelerometer, impedance measurement, Photoplethysmography, PPG sensor, pH sensor, ultrasound sensor, echocardiogram, ultrasound echo, hydrophone, temperature meter, body core temperature, heart pulse rate, Pulse wave properties, glucose sensor, any sensor indicating a change in cardiac output, any sensor indicating blood pressure, any sensor indicating initiation of a dialysis session, acoustic blood pulse wave sensor, electrocardiogram sensor, ultrasonic sensor, or a vibration sensor, any sensor associated with dialysis machine and any combination thereof; further wherein said sensor is adapted to sense at least one parameter selected from a group consisting of movement of said patient, impedance, PPG signal, pH, acoustic signal, pressure, temperature, heart rate, pulse wave properties, glucose level, blood pressure, and any combination thereof.

It is another object of the present invention to provide the implant as defined above, wherein said ultrasonic transducer is an array of transducers.

It is another object of the present invention to provide the implant as defined above, wherein said array is generating said ultrasonic energy of at least one ultrasonic carrier frequency.

It is another object of the present invention to provide the implant as defined above, wherein said at least one on-skin remote controller comprises at least one coil for forming an inductive link and transcutaneous transfer said ultrasonic energy from said at least one on-skin remote controller to said at least one ultrasonic transducer.

It is another object of the present invention to provide the implant as defined above, wherein said at least one on-skin remote controller is adapted to drive said implant by at least one method selected from a group consisting of electromagnetic, ultrasonic, capacitive, induction and any combination thereof.

It is another object of the present invention to provide the implant as defined above, wherein said at least one on-skin remote controller is adapted to collect data from said at least one sensor and perform at least one selected from a group consisting of: (a) monitor said data; (b) adjust the treatment provided to said patient; (c) maintain the treatment provided to said patient as is; and any combination thereof.

It is another object of the present invention to provide the implant as defined above, wherein said data is the shape of the pulse wave signal.

It is another object of the present invention to provide the implant as defined above, wherein said data is the time delay between the ECG signal and the pulse wave signal.

It is another object of the present invention to provide the implant as defined above, wherein said implant is subcutaneously implanted.

It is another object of the present invention to provide the implant as defined above, wherein said implant envelope is coated with Parylene.

It is another object of the present invention to provide the implant as defined above, wherein said at least one transducer is a piezoelectric transducer made of at least one material selected from a group consisting of lead zirconate titanate, lead magnesium niobate-lead titanate, Hard PZT, composite, PZT with non-uniform polarization, PZT with uniform polarization and any combination thereof.

It is another object of the present invention to provide the implant as defined above, wherein said ultrasonic energy may be provided in a continuous manner, or alternatively, in pulses.

It is another object of the present invention to provide the implant as defined above, wherein said at least one on-skin remote controller is adapted to measure heart rate from said at least two pulse waves pulses.

It is another object of the present invention to provide the implant as defined above, wherein analysis of said at least two pulse wave characteristics facilitate alignment of the position of said implant relatively to said at least one vessel or tissue containing flowing blood.

It is another object of the present invention to provide the implant as defined above, wherein at least one of the following is held true (a) said ultrasonic frequency is within 10 kHz-10 MHz; (b) said ultrasonic energy has a peak negative pressure of between about 0.1 MPa and about 2 MPa; and, any combination thereof.

It is another object of the present invention to provide the implant as defined above, wherein said ultrasonic energy is synchronized to the pulse wave signal.

It is another object of the present invention to provide the implant as defined above, wherein said physiological state or change thereof is selected from a group consisting of position of the patient, engagement in physical activity, decrease in NO levels in said at least one vessel or tissue, tissue perfusion, initiation of a physical activity, change in at least one parameter associated with said physical activity, the position of the patient relative to the ground, changes in said position of the patient relative to the ground, application of at least one medical treatment to said patient, changes in application of at least one medical treatment to said patient and any combination thereof.

It is another object of the present invention to provide the implant as defined above, wherein, upon change of said at least one physiological state, at least one notification is being sent to said patient or any care giver thereof.

It is another object of the present invention to provide the implant as defined above, wherein said physiologic effect is selected from the group consisting of: vasodilation, an increase in local ATP, enhance ATP release, an increase in local nitric oxide, enhance nitric oxide release from the vascular endothelium; prolong local nitric oxide effects; enhance nitric oxide release from red blood cells; an alteration in the function of erythrocytes; a modification in oxygen release from hemoglobin, blood temperature increase; vasodilation; prolong local nitric oxide effects; alteration in the function of erythrocytes; modification in oxygen release from hemoglobin; modification in pH of blood;

modulation in the immune response of blood leucocytes; modulation of the coagulation and/or thrombocyte function; modification in the function of heme catalyst enzymes in the blood, improved bioavailability of medication, improved efficiency of a hemodialysis session artery dilation, increased blood perfusion and combinations thereof.

It is another object of the present invention to provide the implant as defined above, wherein at least one of said at least one on-skin remote controller is a wearable by said patient; further wherein said at least one on-skin remote controller being a wearable by said patient is selected from a group consisting of integrated in a wearable sock, shoe, glove, clothes, hats and any combination thereof.

It is another object of the present invention to provide the implant as defined above, wherein said ischemic tissue is selected from a group consisting of the upper limbs and lower limbs, arms, legs and any combination thereof.

It is another object of the present invention to provide the implant as defined above, wherein said at least one on-skin remote controller, adapted to control at least one parameter selected from a group consisting of a phase, an operating frequency, power, intensity, operating amplitude, timing, duration, and any combination thereof of said ultrasonic energy.

It is another object of the present invention to provide the implant as defined above, wherein said at least one on-skin remote controller is in communication with said at least one sensor.

It is another object of the present invention to provide the implant as defined above, wherein upon change of said at least one physiological state of the patient, at least one of the following is being performed (a) said ultrasonic energy is delivered to said at least one vessel or tissue, such that an on-demand treatment is provided; (b) at least one treatment parameter of said ultrasonic energy provision to said at least one vessel or tissue is amended, such that an as-needed treatment is provided; (c) said change is notified; (d) any combination thereof.

It is another object of the present invention to provide the implant as defined above, wherein said sensor is selected from a group consisting of a sensor implanted in said patient, integrated within said at least one casing, integrated within said at least one pig-tail, a sensor being worn by said patient, a remote sensor outside the patient's body, integrated within said at least one on-skin remote controller, and any combination thereof.

It is another object of the present invention to provide the implant as defined above, wherein said at least one ultrasonic transducer functions as acoustic sensor and is adapted to sense at least one acoustic wave generated by said at least one vessel or tissue containing flowing blood.

It is another object of the present invention to provide the implant as defined above, wherein said at least one ultrasonic transducer functions as acoustic sensor and is adapted to sense at least one acoustic wave generated by said at least one vessel or tissue containing flowing blood.

It is another object of the present invention to provide a method of treating a patient, comprising steps of:
a. providing at least one device adapted to be implanted in a patient adjacent to at least one vessel or tissue containing flowing blood, comprising at least one ultrasonic transducer configured to provide ultrasonic energy to be applied to said at least one vessel or tissue containing flowing blood, whereby said ultrasonic energy, when applied, is adapted to cause a physiologic effect in said at least one vessel or tissue;
b. communication said device with at least one on-skin remote controller, positioned externally to the patient, adapted to control said means for applying said ultrasonic energy;
c. implanting said at least one device adjacent to at least one vessel or tissue containing flowing blood;
d. communicating said device with at least one sensor adapted to monitor at least one physiological state of the patient.

It is another object of the present invention to provide a method of treating a patient, comprising steps of:
a. providing at least one implant being adjacent to, or within at least one vessel or tissue containing flowing blood, comprising (i) at least one casing comprising at least one at least one coil for forming an inductive link and transcutaneous transfer said ultrasonic energy from at least one on-skin remote controller to at least one ultrasonic transducer; (ii) at least one sealed pig tail wired or wirelessly connected to said at least one casing, comprising said at least one an ultrasonic transducer configured to couple ultrasonic energy to said at least one vessel or tissue containing flowing blood, whereby said ultrasonic energy, when applied, is adapted to cause a physiologic effect in said at least one vessel or tissue;
b. communication said implant with at least one on-skin remote controller, positioned externally to the patient, adapted to control said means for applying said ultrasonic energy;
c. implanting said at least one implant adjacent to, or within at least one vessel or tissue containing flowing blood;
d. communicating said implant with at least one sensor adapted to monitor at least one physiological state of the patient.

It is another object of the present invention to provide a method of monitoring at least one physiological state of the patient, comprising steps of:
a. providing at least one implant adjacent to at least one vessel or tissue containing flowing blood, comprising at least one pulse wave sensor, adapted to sense at least one acoustic wave generated by said at least one vessel or tissue containing flowing blood and thereby monitor said at least one physiological state of the patient;
b. implanting said at least one implant adjacent to at least one vessel or tissue containing flowing blood;
c. sensing at least one acoustic wave generated by said at least one vessel or tissue containing flowing blood to thereby monitor at least one physiological state of the patient.

It is one object of the present invention to provide an device adapted to be implanted in a patient near, adjacent to, or within at least one vessel or tissue containing flowing blood, comprising means for applying ultrasonic energy (e.g., a transducer) to said at least one vessel or tissue containing flowing blood, whereby said means for applying ultrasonic energy, when applied, is adapted to cause a physiologic effect in said at least one vessel or tissue;
wherein said device is in communication with at least one sensor adapted to monitor at least one physiological state of the patient such that, upon change thereof, at least one of the following is being performed (a) said ultrasonic energy is delivered to said at least one vessel or tissue, such that an on-demand treatment is provided; (b) at least one treatment parameter of said ultrasonic energy provision to said at least one vessel or tissue is amended, such that an as-needed treatment is provided; (c) said change is notified; (d) any combination thereof.

It is another object of the present invention to provide the device as defined above, wherein, upon change of said at least one physiological state, said device is self-activated.

It is another object of the present invention to provide the device as defined above, wherein, upon change of said at least one physiological state, said patient activates said ultrasonic energy or amends said at least one treatment parameter.

It is another object of the present invention to provide the device as defined above, wherein said sensor is selected from a group consisting of accelerometer, impedance measurement, Photoplethysmography, PPG sensor, pH sensor, ultrasound sensor, echocardiogram, ultrasound echo, hydrophone, temperature meter, body core temperature, heart pulse rate, Pulse wave properties, glucose sensor,, any sensor indicating a change in cardiac output, any sensor indicating blood pressure, any sensor indicating initiation of a dialysis session, any sensor associated with dialysis machine and any combination thereof.

It is another object of the present invention to provide the device as defined above, wherein said sensor is adapted to sense at least one parameter selected from a group consisting of movement of said patient, impedance, PPG signal, pH, acoustic signal, pressure, temperature, heart rate, pulse wave properties, glucose level, blood pressure, and any combination thereof.

It is another object of the present invention to provide the device as defined above, wherein said device is in communication with at least one remote controller, positioned externally to the patient, adapted to control said means for applying said ultrasonic energy.

It is another object of the present invention to provide the device as defined above, wherein said sensor is selected from a group consisting of a sensor implanted in said patient, integrated within said device, a sensor being worn by said patient, a remote sensor outside the patient's body, integrated within said at least one controller, and any combination thereof.

It is another object of the present invention to provide the device as defined above, wherein said physiological state or change thereof is selected from a group consisting of position of the patient, engagement in physical activity, decrease in NO levels in said at least one vessel or tissue, tissue perfusion, initiation of a physical activity, change in at least one parameter associated with said physical activity, the position of the patient relative to the ground, changes in said position of the patient relative to the ground, application of at least one medical treatment to said patient, changes in application of at least one medical treatment to said patient and any combination thereof.

It is another object of the present invention to provide the device as defined above, wherein at least one treatment parameter is selected from a group consisting of a phase, an operating frequency, power, intensity, operating amplitude, timing, duration and any combination thereof of said ultrasonic energy.

It is another object of the present invention to provide the device as defined above, wherein, upon change of said at least one physiological state, at least one notification is being sent to said patient or any care giver thereof.

It is another object of the present invention to provide the device as defined above, wherein said device is in communication with at least one battery; said at least one battery is either implanted or positioned externally to the patient.

It is another object of the present invention to provide the device as defined above, wherein said at least one battery is adapted to be wirelessly charged.

It is another object of the present invention to provide the device as defined above, wherein said physiologic effect is selected from the group consisting of: vasodilation; an increase in local nitric oxide; enhance nitric oxide release from the vascular endothelium; prolong local nitric oxide effects; an alteration in the function of erythrocytes; a modification in oxygen release from hemoglobin, blood temperature increase; vasodilation; prolong local nitric oxide effects; alteration in the function of erythrocytes; modification in oxygen release from hemoglobin; modification in pH of blood; modulation in the immune response of blood leucocytes; modulation of the coagulation and/or thrombocyte function; modification in the function of heme catalyst enzymes in the blood, improved bioavailability of medication, improved efficiency of a hemodialysis session artery dilation, increased blood perfusion and combinations thereof

It is another object of the present invention to provide the device as defined above, wherein the ultrasonic energy is provided in frequencies range from about 20 kHz to about 10 MHz.

It is another object of the present invention to provide the device as defined above, wherein the ultrasonic energy is provided by at least one selected from a group consisting of at least one piezoelectric transducer which generates ultrasound energy, at least one passive ferromagnetic element, at least one capacitive micromachined ultrasonic transducer, CMUT, concave transducer, convex transducer and any combination thereof.

It is another object of the present invention to provide the device as defined above, wherein said piezoelectric transducer is made of at least one material selected from a group consisting of lead zirconate titanate, lead magnesium niobate-lead titanate, Hard PZT, composite and any combination thereof.

It is another object of the present invention to provide the device as defined above, wherein said piezoelectric transducer shaded by means of Gaussian apodization.

It is another object of the present invention to provide the device as defined above, wherein said Gaussian apodization is provided by material polarization.

It is another object of the present invention to provide the device as defined above, wherein at least one of said controllers is adapted to charge said device.

It is another object of the present invention to provide the device as defined above, wherein at least one of said controllers is adapted to charge said device by at least one method selected from a group consisting of electromagnetic, ultrasonic, capacitive, induction and any combination thereof.

It is another object of the present invention to provide the device as defined above, wherein at least one of said controllers comprising at least one coil.

It is another object of the present invention to provide the device as defined above, wherein said at least one sensor is adapted to sense at least one pulse wave characteristic, reflected from said device by at least one method selected from a group consisting of (a) passive acoustic sensing of at least one signal reflected from said at least one vessel or tissue; (b) active transmittance of at least one acoustic signal; (c) echo; and any combination thereof.

It is another object of the present invention to provide the device as defined above, wherein device is adapted to sense heart rate from said at least one pulse wave characteristic.

It is another object of the present invention to provide the device as defined above, wherein analysis of said at least one pulse wave characteristic facilitate alignment of the position of said device relatively to said at least one vessel or tissue containing flowing blood.

It is another object of the present invention to provide the device as defined above, wherein said alignment is indicated to either the patient or a care giver thereof.

It is another object of the present invention to provide the device as defined above, wherein said alignment is indicated by at least one indication means selected from a group consisting of audio means, visual means, tactile means and any combination thereof.

It is another object of the present invention to provide the device as defined above, wherein analysis of said at least one pulse wave characteristic indicates said causing of said physiologic effect.

It is another object of the present invention to provide the device as defined above, wherein the ultrasonic energy is provided by an array of piezoelectric transducers, each of which generates ultrasound energy.

It is another object of the present invention to provide the device as defined above, wherein said array of piezoelectric transducers is phased array.

It is another object of the present invention to provide the device as defined above, wherein activation of at least one of said transducers according to a predetermined protocol results in finetuning of said implant's position relative to said at least one vessel so as to align said implant thereto.

It is another object of the present invention to provide the device as defined above, wherein said ultrasonic energy may be provided in a continuous manner, or alternatively, in pulses.

It is another object of the present invention to provide the device as defined above, wherein at least one of said controllers is adapted to collect data from said at least one sensor and perform at least one selected from a group consisting of: (a) monitor said data; (b) amend at least one treatment parameter of the treatment protocol; (c) maintain the treatment provided to said patient as is; and any combination thereof.

It is another object of the present invention to provide the device as defined above, wherein at least one of said controllers is a wearable by said patient.

It is another object of the present invention to provide the device as defined above, wherein at least one of said controllers being a wearable by said patient is selected from a group consisting of integrated in a wearable sock, shoe, glove, clothes, hats and any combination thereof.

It is another object of the present invention to provide the device as defined above, wherein at least one of said controllers is integrated in said patient's environment.

It is another object of the present invention to provide the device as defined above, wherein at least one of said controllers is in communication with at least one selected from a group consisting of CPU, smartphone, and any combination thereof.

It is another object of the present invention to provide the device as defined above, adapted for treatment of pulmonary artery denervation, pulmonary hypertension, ischemic tissues, PAD, CLI, pulmonary artery hypertension, severe asthma patients, improve blood flow to the brain during stroke, increase blood flow to the penis to maintain an erection, enhancement of bioavailability of medications, enhancement of local chemotherapy absorption into a solid tumor by enhancing flow of specific arteries feeding the tumor and any combination thereof.

It is another object of the present invention to provide the device as defined above, wherein said ischemic tissue is selected from a group consisting of the upper limbs and lower limbs, arms, legs and any combination thereof.

It is another object of the present invention to provide the device as defined above, adapted to provide said ultrasonic energy so as to increase blood flow.

It is another object of the present invention to provide the device as defined above, wherein said ultrasonic energy is provided in at least two substantially different frequencies of the carrier signal.

It is another object of the present invention to provide the device as defined above, wherein said device comprises at least one piezoelectric transducer adapted to generate said ultrasonic energy in one of said at least two substantially different frequencies of the carrier signal.

It is another object of the present invention to provide the device as defined above, wherein said device additionally comprises at least one electro-magnetic acoustic transducer, mechanically coupled to said at least one piezoelectric transducer adapted to generate said ultrasonic energy in one of said at least substantially different frequencies of the carrier signal.

It is another object of the present invention to provide the device as defined above, wherein said at least one electro-magnetic acoustic transducer is at least one ferromagnetic sheet.

It is another object of the present invention to provide the device as defined above, wherein one frequency of the carrier signal is in the range selected from about 20 kHz to about 10 MHz.

It is another object of the present invention to provide the device as defined above, wherein the second frequency of the carrier signal is in the range selected from about 20 kHz to about 10 MHz.

It is another object of the present invention to provide the device as defined above, wherein said device is encapsulated in at least one layer of polyetheretherketone, PEEK.

It is another object of the present invention to provide the device as defined above, wherein said device comprises at least one processor, adapted to control at least one parameter selected from a group consisting of a phase, an operating frequency, power, intensity, operating amplitude, timing, duration, and any combination thereof of said ultrasonic energy.

It is another object of the present invention to provide the device as defined above, wherein said at least one processor is in communication with said at least one sensor.

It is another object of the present invention to provide the device as defined above, wherein said at least one processor is adapted to collect data from said at least one sensor and perform at least one selected from a group consisting of: (a) monitor said data; (b) amend the treatment provided to said patient; (c) maintain the treatment provided to said patient as is; and any combination thereof.

It is another object of the present invention to provide a method of treating a patient, comprising steps of:
a. providing at least one implantable device adapted to be implanted in a patient near, adjacent to, or within at least one vessel or tissue containing flowing blood, comprising means for applying ultrasonic energy to said at least one vessel or tissue containing flowing blood, whereby said means for applying ultrasonic energy, when applied, is adapted to cause a physiologic effect in said at least one vessel or tissue;
b. implanting said at least one device adjacent to, or within at least one vessel or tissue containing flowing blood;
c. communicating said device with at least one sensor adapted to monitor at least one physiological state of the patient such that, upon change thereof, at least one of the following is being performed (a) said ultrasonic energy is delivered to said at least one vessel or tissue, such that an on-demand treatment is provided; (b) at least one treatment parameter of said ultrasonic energy provision to said at least one vessel or tissue is amended, such that an as-needed treatment is provided; (c) said change is notified; (d) any combination thereof; thereby treating said patient.

It is another object of the present invention to provide the method as defined above, wherein, upon change of said at least one physiological state, said device is self-activated.

It is another object of the present invention to provide the method as defined above, wherein, upon change of said at least one physiological state, said patient activates said ultrasonic energy or amends said at least one treatment parameter.

It is another object of the present invention to provide the method as defined above, wherein said sensor is selected from a group consisting of accelerometer, impedance measurement, Photoplethysmography, PPG sensor, pH sensor, ultrasound sensor, echocardiogram, ultrasound echo, hydrophone, temperature meter, body core temperature, heart pulse rate, Pulse wave properties, glucose sensor,, any sensor indicating a change in cardiac output, any sensor indicating blood pressure, any sensor indicating initiation of a dialysis session, any sensor associated with dialysis machine and any combination thereof.

It is another object of the present invention to provide the method as defined above, wherein said sensor is adapted to sense at least one parameter selected from a group consisting of movement of said patient, impedance, PPG signal, pH, acoustic signal, pressure, temperature, heart rate, pulse wave properties, glucose level, blood pressure, and any combination thereof.

It is another object of the present invention to provide the method as defined above, wherein said device is in communication with at least one remote controller, positioned externally to the patient, adapted to control said means for applying said ultrasonic energy.

It is another object of the present invention to provide the method as defined above, wherein said sensor is selected from a group consisting of a sensor implanted in said patient, integrated within said device, a sensor being worn by said patient, a remote sensor outside the patient's body, integrated within said at least one controller, and any combination thereof.

It is another object of the present invention to provide the method as defined above, wherein said physiological state or change thereof is selected from a group consisting of position of the patient, engagement in physical activity, decrease in NO levels in said at least one vessel or tissue, tissue perfusion, initiation of a physical activity, change in at least one parameter associated with said physical activity, the position of the patient relative to the ground, changes in said position of the patient relative to the ground, application of at least one medical treatment to said patient, changes in application of at least one medical treatment to said patient and any combination thereof.

It is another object of the present invention to provide the method as defined above, wherein at least one treatment parameter is selected from a group consisting of a phase, an operating frequency, power, intensity, operating amplitude, timing, duration and any combination thereof of said ultrasonic energy.

It is another object of the present invention to provide the method as defined above, wherein, upon change of said at least one physiological state, at least one notification is being sent to said patient or any care giver thereof.

It is another object of the present invention to provide the method as defined above, wherein said device is in communication with at least one battery; said at least one battery is either implanted or positioned externally to the patient.

It is another object of the present invention to provide the method as defined above, wherein said at least one battery is adapted to be wirelessly charged.

It is another object of the present invention to provide the method as defined above, wherein said physiologic effect is selected from the group consisting of: vasodilation; an increase in local nitric oxide; enhance nitric oxide release from the vascular endothelium; prolong local nitric oxide effects; an alteration in the function of erythrocytes; a modification in oxygen release from hemoglobin, blood temperature increase; vasodilation; prolong local nitric oxide effects; alteration in the function of erythrocytes; modification in oxygen release from hemoglobin; modification in pH of blood; modulation in the immune response of blood leucocytes; modulation of the coagulation and/or thrombocyte function; modification in the function of heme catalyst enzymes in the blood, improved bioavailability of medication, improved efficiency of a hemodialysis session artery dilation, increased blood perfusion and combinations thereof

It is another object of the present invention to provide the method as defined above, wherein the ultrasonic energy is provided in frequencies range from about 20 kHz to about 10 MHz.

It is another object of the present invention to provide the method as defined above, wherein the ultrasonic energy is provided by at least one selected from a group consisting of at least one piezoelectric transducer which generates ultrasound energy, at least one passive ferromagnetic element, at least one capacitive micromachined ultrasonic transducer, CMUT, concave transducer, convex transducer and any combination thereof.

It is another object of the present invention to provide the method as defined above, wherein said piezoelectric transducer is made of at least one material selected from a group consisting of lead zirconate titanate, lead magnesium niobate-lead titanate, Hard PZT, composite and any combination thereof.

It is another object of the present invention to provide the method as defined above, wherein said piezoelectric transducer shaded by means of Gaussian apodization.

It is another object of the present invention to provide the method as defined above, wherein said Gaussian apodization is provided by material polarization.

It is another object of the present invention to provide the method as defined above, wherein at least one of said controllers is adapted to charge said device.

It is another object of the present invention to provide the method as defined above, wherein at least one of said controllers is adapted to charge said device by at least one method selected from a group consisting of electromagnetic, ultrasonic, capacitive, induction and any combination thereof.

It is another object of the present invention to provide the method as defined above, wherein at least one of said controllers comprising at least one coil.

It is another object of the present invention to provide the method as defined above, wherein said at least one sensor is adapted to sense at least one pulse wave characteristic, reflected from said device by at least one method selected from a group consisting of (a) passive acoustic sensing of at least one signal reflected from said at least one vessel or tissue; (b) active transmittance of at least one acoustic signal; (c) echo; and any combination thereof.

It is another object of the present invention to provide the method as defined above, wherein device is adapted to sense heart rate from said at least one pulse wave characteristic.

It is another object of the present invention to provide the method as defined above, wherein analysis of said at least one pulse wave characteristic facilitate alignment of the position of said device relatively to said at least one vessel or tissue containing flowing blood.

It is another object of the present invention to provide the method as defined above, wherein said alignment is indicated to either the patient or a care giver thereof.

It is another object of the present invention to provide the method as defined above, wherein said alignment is indicated by at least one indication means selected from a group consisting of audio means, visual means, tactile means and any combination thereof.

It is another object of the present invention to provide the method as defined above, wherein analysis of said at least one pulse wave characteristic indicates said causing of said physiologic effect.

It is another object of the present invention to provide the method as defined above, wherein the ultrasonic energy is provided by an array of piezoelectric transducers, each of which generates ultrasound energy.

It is another object of the present invention to provide the method as defined above, wherein said array of piezoelectric transducers is phased array.

It is another object of the present invention to provide the method as defined above, wherein activation of at least one of said transducers according to a predetermined protocol results in finetuning of said implant's position relative to said at least one vessel so as to align said implant thereto.

It is another object of the present invention to provide the method as defined above, wherein said ultrasonic energy may be provided in a continuous manner, or alternatively, in pulses.

It is another object of the present invention to provide the method as defined above, wherein at least one of said controllers is adapted to collect data from said at least one sensor and perform at least one selected from a group consisting of: (a) monitor said data; (b) amend at least one treatment parameter of the treatment protocol; (c) maintain the treatment provided to said patient as is; and any combination thereof.

It is another object of the present invention to provide the method as defined above, wherein at least one of said controllers is a wearable by said patient.

It is another object of the present invention to provide the method as defined above, wherein at least one of said controllers being a wearable by said patient is selected from a group consisting of integrated in a wearable sock, shoe, glove, clothes, hats and any combination thereof.

It is another object of the present invention to provide the method as defined above, wherein at least one of said controllers is integrated in said patient's environment.

It is another object of the present invention to provide the method as defined above, wherein at least one of said controllers is in communication with at least one selected from a group consisting of CPU, smartphone, and any combination thereof.

It is another object of the present invention to provide the method as defined above, adapted for treatment of pulmonary artery denervation, pulmonary hypertension, ischemic tissues, PAD, CLI, pulmonary artery hypertension, severe asthma patients, improve blood flow to the brain during stroke, increase blood flow to the penis to maintain an erection, enhancement of bioavailability of medications, enhancement of local chemotherapy absorption into a solid tumor by enhancing flow of specific arteries feeding the tumor and any combination thereof.

It is another object of the present invention to provide the method as defined above, wherein said ischemic tissue is selected from a group consisting of the upper limbs and lower limbs, arms, legs and any combination thereof.

It is another object of the present invention to provide the method as defined above, adapted to provide said ultrasonic energy so as to increase blood flow.

It is another object of the present invention to provide the method as defined above, wherein said ultrasonic energy is provided in at least two substantially different frequencies of the carrier signal.

It is another object of the present invention to provide the method as defined above, wherein said device comprises at least one piezoelectric transducer adapted to generate said ultrasonic energy in one of said at least two substantially different frequencies of the carrier signal.

It is another object of the present invention to provide the method as defined above, wherein said device additionally comprises at least one electro-magnetic acoustic transducer, mechanically coupled to said at least one piezoelectric transducer adapted to generate said ultrasonic energy in one of said at least substantially different frequencies of the carrier signal.

It is another object of the present invention to provide the method as defined above, wherein said at least one electro-magnetic acoustic transducer is at least one ferromagnetic sheet.

It is another object of the present invention to provide the method as defined above, wherein one frequency of the carrier signal is in the range selected from about 20 kHz to about 10 MHz.

It is another object of the present invention to provide the method as defined above, wherein the second frequency of the carrier signal is in the range selected from about 20 kHz to about 10 MHz.

It is another object of the present invention to provide the method as defined above, wherein said device is encapsulated in at least one layer of polyetheretherketone, PEEK.

It is another object of the present invention to provide the method as defined above, wherein said device comprises at least one processor, adapted to control at least one parameter selected from a group consisting of a phase, an operating frequency, power, intensity, operating amplitude, timing, duration, and any combination thereof of said ultrasonic energy.

It is another object of the present invention to provide the method as defined above, wherein said at least one processor is in communication with said at least one sensor.

It is another object of the present invention to provide the method as defined above, wherein said at least one processor is adapted to collect data from said at least one sensor and perform at least one selected from a group consisting of: (a) monitor said data; (b) amend the treatment provided to said patient; (c) maintain the treatment provided to said patient as is; and any combination thereof.

It is another object of the present invention to provide the method as defined above, additionally comprising step of orienting at least two devices at the same position relative to said at least one vessel or tissue containing flowing blood.

It is another object of the present invention to provide the method as defined above, additionally comprising step of orienting at least two devices at a substantially different position relative to said at least one vessel or tissue containing flowing blood.

It is another object of the present invention to provide the method as defined above, additionally comprising step of enabling focusing said ultrasonic energy on said at least one vessel or tissue by means of said positioning of said at least two device.

It is another object of the present invention to provide the method as defined above, additionally comprising step of enabling communication between at least two of devices.

It is another object of the present invention to provide a system comprising a plurality of implantable ultrasonic devices, at least one of which is to be implanted in a patient near, adjacent to, or within at least one vessel or tissue containing flowing blood, comprising means for applying ultrasonic energy to said at least one vessel or tissue containing flowing blood, whereby said means for applying ultrasonic energy, when applied, is adapted to cause a physiologic effect in said at least one vessel or tissue; wherein at least one of said devices is in communication with at least one sensor adapted to monitor at least one physiological state of the patient such that, upon change thereof, at least one of the following is being performed (a) said ultrasonic energy is delivered to said at least one vessel or tissue, such that an on-demand treatment is provided; (b) at least one treatment parameter of said ultrasonic energy provision to said at least one vessel or tissue is amended, such that an as-needed treatment is provided; (c) said change is notified; (d) any combination thereof.

It is another object of the present invention to provide the system as defined above, wherein, upon change of said at least one physiological state, at least one of said devices is self-activated.

It is another object of the present invention to provide the system as defined above, wherein, upon change of said at least one physiological state, said patient activates said ultrasonic energy or amends said at least one treatment parameter.

It is another object of the present invention to provide the system as defined above, wherein said sensor is selected from a group consisting of accelerometer, impedance measurement, Photoplethysmography, PPG sensor, pH sensor, ultrasound sensor, echocardiogram, ultrasound echo, hydrophone, temperature meter, body core temperature, heart pulse rate, Pulse wave properties, glucose sensor,, any sensor indicating a change in cardiac output, any sensor indicating blood pressure, any sensor indicating initiation of a dialysis session, any sensor associated with dialysis machine and any combination thereof.

It is another object of the present invention to provide the system as defined above, wherein said sensor is adapted to sense at least one parameter selected from a group consisting of movement of said patient, impedance, PPG signal, pH, acoustic signal, pressure, temperature, heart rate, pulse wave properties, glucose level, blood pressure, and any combination thereof.

It is another object of the present invention to provide the system as defined above, wherein at least one said device is in communication with at least one remote controller, positioned externally to the patient, adapted to control said means for applying said ultrasonic energy.

It is another object of the present invention to provide the system as defined above, wherein said sensor is selected from a group consisting of a sensor implanted in said patient, integrated within at least one of said devices, a sensor being worn by said patient, a remote sensor outside the patient's body, integrated within said at least one controller, and any combination thereof.

It is another object of the present invention to provide the system as defined above, wherein said physiological state or change thereof is selected from a group consisting of position of the patient, engagement in physical activity, decrease in NO levels in said at least one vessel or tissue, tissue perfusion, initiation of a physical activity, change in at least one parameter associated with said physical activity, the position of the patient relative to the ground, changes in said position of the patient relative to the ground, application of at least one medical treatment to said patient, changes in application of at least one medical treatment to said patient and any combination thereof.

It is another object of the present invention to provide the system as defined above, wherein at least one treatment parameter is selected from a group consisting of a phase, an operating frequency, power, intensity, operating amplitude, timing, duration and any combination thereof of said ultrasonic energy.

It is another object of the present invention to provide the system as defined above, wherein, upon change of said at least one physiological state, at least one notification is being sent to said patient or any care giver thereof.

It is another object of the present invention to provide the system as defined above, wherein at least one of said devices is in communication with at least one battery; said at least one battery is either implanted or positioned externally to the patient.

It is another object of the present invention to provide the system as defined above, wherein said at least one battery is adapted to be wirelessly charged.

It is another object of the present invention to provide the system as defined above, wherein said physiologic effect is selected from the group consisting of: vasodilation; an increase in local nitric oxide; enhance nitric oxide release from the vascular endothelium; prolong local nitric oxide effects; an alteration in the function of erythrocytes; a modification in oxygen release from hemoglobin, blood temperature increase; vasodilation; prolong local nitric oxide effects; alteration in the function of erythrocytes; modification in oxygen release from hemoglobin; modification in pH of blood; modulation in the immune response of blood leucocytes; modulation of the coagulation and/or thrombocyte function; modification in the function of heme catalyst enzymes in the blood, improved bioavailability of medication, improved efficiency of a hemodialysis session artery dilation, increased blood perfusion and combinations thereof

It is another object of the present invention to provide the system as defined above, wherein the ultrasonic energy is provided in frequencies range from about 20 kHz to about 10 MHz.

It is another object of the present invention to provide the system as defined above, wherein the ultrasonic energy is provided by at least one selected from a group consisting of at least one piezoelectric transducer which generates ultrasound system, at least one passive ferromagnetic element, at least one capacitive micromachined ultrasonic transducer, CMUT, concave transducer, convex transducer and any combination thereof.

It is another object of the present invention to provide the system as defined above, wherein said piezoelectric transducer is made of at least one material selected from a group consisting of lead zirconate titanate, lead magnesium niobate-lead titanate, Hard PZT, composite and any combination thereof.

It is another object of the present invention to provide the system as defined above, wherein said piezoelectric transducer shaded by means of Gaussian apodization.

It is another object of the present invention to provide the system as defined above, wherein said Gaussian apodization is provided by material polarization.

It is another object of the present invention to provide the system as defined above, wherein at least one of said controllers is adapted to charge at least one of said devices.

It is another object of the present invention to provide the system as defined above, wherein at least one of said controllers is adapted to charge at least one of said devices by at least one method selected from a group consisting of electromagnetic, ultrasonic, capacitive, induction and any combination thereof.

It is another object of the present invention to provide the system as defined above, wherein at least one of said controllers comprising at least one coil.

It is another object of the present invention to provide the system as defined above, wherein said at least one sensor is adapted to sense at least one pulse wave characteristic, reflected from at least one of said devices by at least one method selected from a group consisting of (a) acoustic sensing; (b) echo and any combination thereof.

It is another object of the present invention to provide the system as defined above, wherein at least one of said devices is adapted to sense heart rate from said at least one pulse wave characteristic.

It is another object of the present invention to provide the system as defined above, wherein analysis of said at least one pulse wave characteristic facilitate alignment of the position of at least one of said devices relatively to said at least one vessel or tissue containing flowing blood.

It is another object of the present invention to provide the system as defined above, wherein said alignment is indicated to either the patient or a care giver thereof.

It is another object of the present invention to provide the system as defined above, wherein said alignment is indicated by at least one indication means selected from a group consisting of audio means, visual means, tactile means and any combination thereof.

It is another object of the present invention to provide the system as defined above, wherein analysis of said at least one pulse wave characteristic indicates said causing of said physiologic effect.

It is another object of the present invention to provide the system as defined above, wherein the ultrasonic energy is provided by an array of piezoelectric transducers, each of which generates ultrasound energy.

It is another object of the present invention to provide the system as defined above, wherein said array of piezoelectric transducers is phased array.

It is another object of the present invention to provide the system as defined above, wherein activation of at least one of said transducers according to a predetermined protocol results in finetuning of said implant's position relative to said at least one vessel so as to align said implant thereto.

It is another object of the present invention to provide the system as defined above, wherein said ultrasonic energy may be provided in a continuous manner, or alternatively, in pulses.

It is another object of the present invention to provide the system as defined above, wherein at least one of said controllers is adapted to collect data from said at least one sensor and perform at least one selected from a group consisting of: (a) monitor said data; (b) amend at least one treatment parameter of the treatment protocol; (c) maintain the treatment provided to said patient as is; and any combination thereof.

It is another object of the present invention to provide the system as defined above, wherein at least one of said controllers is a wearable by said patient.

It is another object of the present invention to provide the system as defined above, wherein at least one of said controllers being a wearable by said patient is selected from a group consisting of integrated in a wearable sock, shoe, glove, clothes, hats and any combination thereof.

It is another object of the present invention to provide the system as defined above, wherein at least one of said controllers is integrated in said patient's environment.

It is another object of the present invention to provide the system as defined above, wherein at least one of said controllers is in communication with at least one selected from a group consisting of CPU, smartphone, and any combination thereof.

It is another object of the present invention to provide the system as defined above, adapted for treatment of pulmonary artery denervation, pulmonary hypertension, ischemic tissues, PAD, CLI, pulmonary artery hypertension, severe asthma patients, improve blood flow to the brain during stroke, increase blood flow to the penis to maintain an erection, enhancement of bioavailability of medications, enhancement of local chemotherapy absorption into a solid tumor by enhancing flow of specific arteries feeding the tumor and any combination thereof.

It is another object of the present invention to provide the system as defined above, wherein said ischemic tissue is selected from a group consisting of the upper limbs and lower limbs, arms, legs and any combination thereof.

It is another object of the present invention to provide the system as defined above, adapted to provide said ultrasonic energy so as to increase blood flow.

It is another object of the present invention to provide the system as defined above, wherein said ultrasonic energy is provided in at least two substantially different frequencies of the carrier signal.

It is another object of the present invention to provide the system as defined above, wherein at least one of said devices comprises at least one piezoelectric transducer adapted to generate said ultrasonic energy in one of said at least two substantially different frequencies of the carrier signal.

It is another object of the present invention to provide the system as defined above, wherein at least one of said devices additionally comprises at least one electro-magnetic acoustic transducer, mechanically coupled to said at least one piezoelectric transducer adapted to generate said ultrasonic energy in one of said at least substantially different frequencies of the carrier signal.

It is another object of the present invention to provide the system as defined above, wherein said at least one electro-magnetic acoustic transducer is at least one ferromagnetic sheet.

It is another object of the present invention to provide the system as defined above, wherein one frequency of the carrier signal is in the range selected from about 20 kHz to about 10 MHz.

It is another object of the present invention to provide the system as defined above, wherein the second frequency of the carrier signal is in the range selected from about 20 kHz to about 10 MHz.

It is another object of the present invention to provide the system as defined above, wherein at least one of said devices is encapsulated in at least one layer of polyetheretherketone, PEEK.

It is another object of the present invention to provide the system as defined above, wherein at least one of said devices comprises at least one processor, adapted to control at least one parameter selected from a group consisting of a phase, an operating frequency, power, intensity, operating amplitude, timing, duration, and any combination thereof of said ultrasonic energy.

It is another object of the present invention to provide the system as defined above, wherein said at least one processor is in communication with said at least one sensor.

It is another object of the present invention to provide the system as defined above, wherein said at least one processor is adapted to collect data from said at least one sensor and perform at least one selected from a group consisting of: (a) monitor said data; (b) amend the treatment provided to said patient; (c) maintain the treatment provided to said patient as is; and any combination thereof.

It is another object of the present invention to provide the system as defined above, wherein all of said plurality of implants are oriented at the same position relative to said at least one vessel or tissue containing flowing blood.

It is another object of the present invention to provide the system as defined above, wherein each of said plurality of implants is oriented at a substantially different position relative to said at least one vessel or tissue containing flowing blood, relative to another one of said plurality of implants.

It is still an object of the present invention to provide the system as defined above, wherein said position of said plurality of implants is adapted to enable focusing of said energy on said at least one vessel or tissue containing flowing blood.

It is lastly an object of the present invention to provide the system as defined above, wherein at least two of said plurality of implantable devices are in communication with each other another.

Various other objects, aspects and advantages of the present inventive disclosure can be obtained from a study of the specification, the drawings, and the appended claims.

### BRIEF DESCRIPTION OF THE FIGURES

For simplicity and clarity of illustration, elements shown in the figures have not necessarily been drawn to scale. For example, the dimensions of some of the elements may be exaggerated relative to other elements for clarity of presentation. Furthermore, reference numerals may be repeated among the figures to indicate corresponding or analogous elements.

The figures are listed below.

FIGS. 1A-1B illustrates a general overview of the system, in which the ultrasonic transducer is located within the implant. The system contains various sensors such as thermal sensor for protection, that enables to limit the temperature of the implant envelope below a predetermined safety level.

FIG. 2 illustrates a general overview of the system where the ultrasonic transducer is located at the pig tail cable emerging from the implant case, positioned close to the artery.

FIG. 3 illustrates a typical pulsed mode ultrasonic energy coupled to the tissue. the parameters of the ultrasonic waves such as peak negative pressure, carrier frequency, pulse duration, and pulse repetition frequency may be changed according to the data collected from the sensors.

FIG. 4 illustrates a general realization of the ultrasonic implant of the system.

FIG. 5 illustrates the blood pulse wave sensing by the implant. The pulse wave causes an acoustic signal. When passing in the artery against the implant, the acoustic signal is detected by the implant.

FIG. 6 illustrates a typical realization of the on skin control unit. The on skin unit may be in the form of a sleeve, wearable device etc.

FIG. 7 illustrates a possible transmitting method of the implant, using load key modulation

FIG. 8 illustrates a time delay measurement between an ECG and pulse wave signals.

FIGS. 9-11 schematically illustrate different embodiments of the present invention;

FIG. 12 illustrates an embodiment in which multilayer acoustic matching are implemented;

FIG. 13 illustrates an embodiment in which a passive implant is provided;

FIG. 14 illustrates an embodiment in which the NO induction is provided by utilizing an already implanted stent.

FIG. 15 illustrates an embodiment in which the ultrasonic energy is provided in at least two substantially different frequencies of the carrier signal.

### DETAIL DESCRIPTION OF SPECIFIC EMBODIMENTS OF THE INVENTION

The present invention, in some embodiments thereof, relates to a subcutaneous implantable device that delivers therapeutic energy to a patient. More specifically relates to implantable ultrasonic devices adapted to affect vascular endothelium to induce Nitric Oxide, NO, and ATP release. Thus, by applying ultrasonic energy the blood vessel or tissue, the stimulating cavitation and shear stress promotes angiogenesis within the patient. Furthermore, the present disclosure relates generally to the field of pulmonary artery denervation in the treatment of pulmonary hypertension and ischemic tissues.

Several cardiovascular diseases still lack an effective treatment with substantial improvement of prognosis. One of the most prominent is primary pulmonary hypertension, PH, a disease in which elevated vascular resistance in the lungs often leads to right heart failure and premature death. Therapy using endothelin receptor antagonist has raised hope for clinicians and patients. A major pathophysiologic mechanism for pulmonary hypertension is the lack of nitric oxide in the pulmonary vascular vessels. Thus, the present invention discloses a plurality of implants adapted to apply either ultrasonic or vibrational energy to induce NO release for the treatment of pulmonary hypertension, PH.

Another prominent use of NO and ATP induction is in ischemic tissue. Thus, the present invention also relates to improving NO and ATP release in ischemic tissue by applying ultrasound to the tissue under conditions effective to increase NO and ATP release in said ischemic tissue (e.g., treating ischemic limbs or tissue affected by peripheral arterial disease). Peripheral arterial disease is the most common form of atherosclerosis that affects many people worldwide. As a result of such disease, many people experience pain during physical activity (e.g., walking). Thus, the present invention discloses a subcutaneous implantable adapted to apply either ultrasonic or vibrational energy to induce NO and ATP release for the treatment of ischemic tissue.

It is one object of the present invention to provide an ultrasound subcutaneous implant which can locally dilate the targeted artery and increase downstream perfusion. It is another object of the present invention to provide system comprising a plurality of implantable devices, to be implanted in a patient near, adjacent to, or within at least one vessel or tissue containing flowing blood, each of said plurality of implantable devices comprising means for applying at least one source of energy (e.g., an ultrasonic transducer) to said within at least one vessel or tissue containing flowing blood, whereby said source of energy, when applied, is adapted to cause a physiologic effect in said at least one vessel or tissue; wherein at least one of said implantable devices is in communication with at least one remote controller (e.g., worn by the patient), positioned externally to the patient adapted to program and control said means for applying said at least one source of energy.

Such wearable controller could be integrated in a wearable sock, shoe, glove, clothes, hats, or integrated in said patient's environment (e.g., furniture; for example, chairs, sofa, bed etc.) and any combination thereof.

Thus, each of the implants can locally dilate the targeted artery and increase downstream perfusion. The mechanism of action involves acoustic energy that is absorbed by endothelial cells and erythrocytes resulting in release of adenosine triphosphate (ATP) and nitric oxide (NO) into the blood stream. The NO will dilate the artery and increase local blood flow and perfusion. In PAD/CLI patients, suffering from claudication, the implant will be placed a few centimeters (e.g., 2-3 cm) upstream from the ischemic region to locally increase tissue perfusion with the goal of lowering the hypoxia pain during walking, physical exercising or in rest. It should be noted that, the implant's positioning could initially be calculated by the half-life of NO and the flow velocity in the required specific vessel, such that tailored positioning could be provided to ensure the most effective vasodilation effect is achieved for the desired distance.

According to one embodiment, at least one of said plurality of implantable devices is in communication with at least one battery, either implanted or positioned externally to the patient.

According to one embodiment, the implant comprises or is in communication with at least one sensor. Said sensor is adapted to monitor at least one physiological state of the patient. Once a change is detected the device is either self-activated to apply said energy (and thereby to induce NO release) or to notify the patient or any predefined care giver of the patient of said change and either the patient or the care giver activates the device.

According to one embodiment, the sensor is selected from a group consisting of a sensor implanted in said patient, a sensor being worn by said patient, a remote sensor outside the patient's body and any combination thereof. For example, the sensor can also be outside the body (e.g. a wearable) transmitting to the implant or it can be implanted elsewhere in the body. For example, a pacemaker that senses activity can also send a signal to our implant so both device work in concert and the heart rate increases while NO is released. Another example can be other sensors that are used in cardiology (e.g. heart failure implantable sensors like the BSC HeartLogic and the Abbot CardioMems).

According to other embodiments of the present invention, the implant is in communication with at least one controller; said controller is adapted to receive information associated with at least one physiological state of the patient. According to another embodiment, the controller is in communication with at least one sensor, adapted to sense information associated with at least one physiological state of the patient.

As used herein with reference to quantity or value, the term “about” means “within ±10% of”.

The terms “comprises”, “comprising”, “includes”, “including”, “has”, “having” and their conjugates mean “including but not limited to”.

Throughout this application, embodiments of this invention may be presented with reference to a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

The present invention discloses an implant device that comprises at least one transducer (e.g., piezoelectric) which generates either ultrasonic energy or vibrational energy to mimic application of shear forces on the endothelium cell to induce NO and ATP production and release.

The implant would be implanted in proximity to ischemic tissues (namely, the upper limbs and lower limbs, i.e., the arms and legs).

Activation of said implant device to provide vibrations or ultrasonic energy could primally affect local NO release from endothelium cells. Additionally, such device could also induce ATP release. It should be noted that, it is within the scope of the present invention to provide the device constructed and arranged to cause any other physiologic effect selected from the group consisting of: blood temperature increase; vasodilation; prolong local nitric oxide effects; alteration in the function of erythrocytes; modification in oxygen release from hemoglobin; modification in pH of blood; modulation in the immune response of blood leucocytes; modulation of the coagulation and/or thrombocyte function; modification in the function of heme catalyst enzymes in the blood, improved bioavailability of medication, improved efficiency of a hemodialysis session and combinations of these.

According to another embodiment, the device would be activated from outside the patient's body. Thus, according to this embodiment, a remote controller being positioned externally to the patient is in communication with the device, adapted to activate the same and can be used by the patient himself or a ‘health care’ provider to program and control the source of energy and thus, the treatment protocol delivered to the patient (e.g., increase/decrease intensity of the energy, time, level of energy, source of energy etc.).

According to one embodiment, an ‘on-demand’ device/system will be provided. It is noted that the ‘on demand’ device will be activated only when needed (‘on-demand’ basis). According to this embodiment, the device will be integrated/incommunication with at least one sensor that monitors physiological state of the patient such that, upon change thereof, the device is activated or the treatment protocol of the device is amended.

According to this embodiment, the sensors of the ‘on-demand’ device could be selected from a group consisting of accelerometer, impedance measurement, Photoplethysmography, PPG sensor, pH sensor, Ultrasound sensor, hydrophone, temperature meter, manual activation, any sensor indicating a change in cardiac output, any sensor indicating blood pressure, any sensor indicating initiation of a dialysis session, any sensor associated with dialysis machine and any combination thereof.

Each of the sensors is adapted to monitor the physiological state of the patient and provide treatment accordingly. For example, accelerometer will be used to indicate if the position of the patient (e.g., when the patient is lying down, standing, walking etc.). Upon sense of a change (e.g., when the patient starts to walk) the device is activated. In other words, the device is activated ‘on demand’ (when the patient engages in physical activity, e.g., walking) to induce NO release (and thus, increase oxidation levels, tissue perfusion in the tissue and decrease any pain associated therewith).

Thus, it is another object of the present invention to provide the implantable device as defined above, wherein said sensor is selected so that indication of level of physical activity or changes thereof can be estimated for example initiation of walking or change in change in walking parameter such as speed, stride length, cadence or moving to standing from seating or laying down or moving from walking to running.

It is within the scope of this invention, wherein said sensor is selected from a group consisting of accelerometer, impedance measurement, Photoplethysmography, PPG sensor, pH sensor, Ultrasound sensor, hydrophone, temperature meter, manual activation, any sensor indicating a change in cardiac output, any sensor indicating blood pressure, any sensor indicating initiation of a dialysis session, any sensor associated with dialysis machine and any combination thereof.

Another example is the integration of impedance measurement sensor. According to this embodiment, vasodilation reduces local impedance around the vessel due to higher fluid volume compared to solid tissue. Thus, such impedance sensor will provide indication as for the NO/oxidation level in the tissue or blood vessel and therefrom, input could be retrieved as for the required activation of the device or any amendment needed to the treatment protocol of the device (increased US intensity, treatment duration time etc.).

According to another embodiment, the subcutaneous implant or wearable device will comprise an ultrasound transducers (and, optionally, at least one ultrasound receivers), rechargeable battery and an array of sensors to measure physiological parameters (e.g. patient activity or local tissue perfusion) and enable an ‘on demand’ treatment. For example, the device can sense walking and applies therapy at the right timing to minimize pain. It should be noted that local tissue perfusion and oxidation level can be measured by impedance, PPG, ultrasound echo and any combination thereof.

As described above, according to one embodiment of the present invention the device is in communication with an external charging unit for wirelessly charging the implant. According to another embodiment, the external unit will enable downloading the data collected by the sensors.

The charging unit can be designed as a wearable unit onto the patient. According to one embodiment, the charging unit will be worn once every predetermined time (e.g., once a day). According to another embodiment, the charging unit will be placed in a fixed location or integrated within the patient natural environment (e.g., furniture, e.g., under the bed's covers).

According to another embodiment of the present invention a data controller will be provided (e.g., in the form of a cellular application) enabling both clinicians and patient to control the device activity. The controller will be connected the data for monitoring and based thereupon amend/maintain the treatment protocol thereof.

It should be noted that it is well within the scope of the present invention to disclose a system, comprises a plurality of such device, being in at least partial communication with each other, at least one of which that comprises at least one piezoelectric transducer or passive ferromagnetic element, which generates either ultrasonic energy or vibrational energy to mimic application of shear forces on the endothelium cell to induce NO production and release.

According to one embodiment, the piezoelectric transducer is made of at least one material selected from a group consisting of lead zirconate titanate, lead magnesium niobate-lead titanate, Hard PZT, composite and any combination thereof.

The implant would be subcutaneously implanted in proximity to ischemic tissues (namely, the upper limbs and lower limbs, i.e., the arms and legs). Alternatively, the wearable device would be worn in proximity to ischemic tissues.

Activation of said implant to provide vibrations or ultrasonic energy could primally affect local NO release from endothelium cells. Additionally, such implant could induce ATP release. It should be noted that, it is within the scope of the present invention to provide the device constructed and arranged to cause any other physiologic effect selected from the group consisting of: blood temperature increase; vasodilation; prolong local nitric oxide effects; alteration in the function of erythrocytes; modification in oxygen release from hemoglobin; modification in pH of blood; modulation in the immune response of blood leucocytes; modulation of the coagulation and/or thrombocyte function; modification in the function of heme catalyst enzymes in the blood, improved bioavailability of medication, improved efficiency of a hemodialysis session and combinations of these.

According to one embodiment of the present invention, enhancement of bioavailability of medication could be utilized e.g., in chemotherapy. According to this embodiment, the device could be positioned in the vicinity of a solid tumor. Activation thereof during systemic chemotherapy session will result in enhancement of blood flow to the tumor when the chemotherapy is provided.

According to another embodiment, the device would be activated from outside the patient's body. Thus, according to this embodiment, a remote controller that comprise an electronic communication device external to the patient which is in communication with at least one of the implants, adapted to activate the same and can be used by a ‘health care’ provider to program and control the source of energy and thus, the treatment protocol delivered to the patient (e.g., increase/decrease amount, time, level of energy, source of energy etc.).

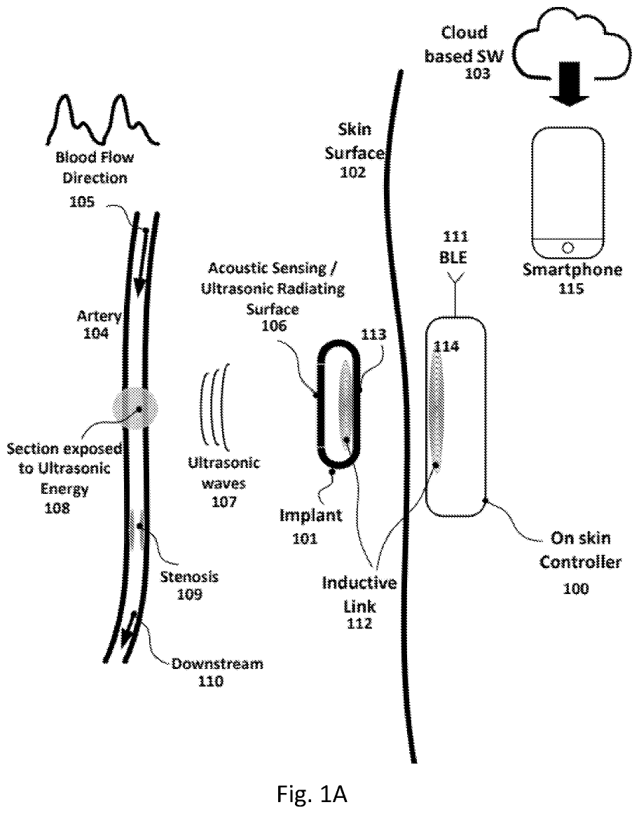

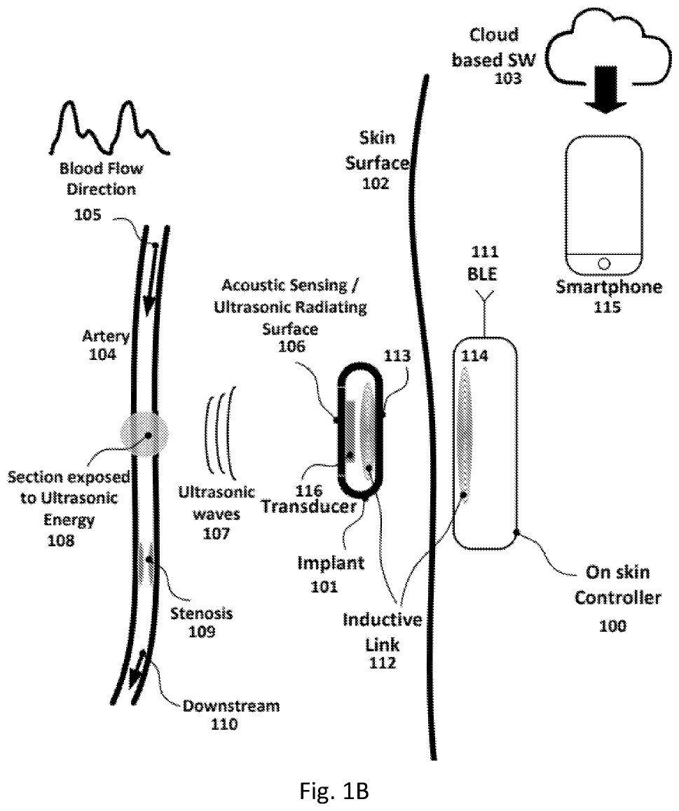

Reference is now made to FIG. 1A illustrating a general overview of the system (the implant and the on-skin controller) which includes the implant 101 and the on-skin unit 100.

As seen in FIG. 1A, in the implant 101, a coil is attached to its back surface 113, forming an inductive link 112 with the on-skin inductor 114. The inductive link may be utilized both for power transfer from the on-skin unit 108 to the implant 101, through the skin 102, and as a communication channel for exchanging data and control between the on-skin unit 100 and the implant 101.

In some embodiments, the on-skin unit 100, may communicate with external host via a wireless link 111, such as, but not limited to, Bluetooth Low Energy, BLE.

As seen in the figure, a blood pulse 105 flows in a blood vessel (e.g., artery) 104. A stenosis 109 limits the flow in the blood vessel downstream 110. To augment the blood flow, an implant 101 generates ultrasonic energy 107, that is directed toward the blood vessel 104. The system may be programmed to emit ultrasound energy automatically, or on-command from the patient. Consequently, the blood vessel section 108 (that may be close to the stenosis section 109) is exposed to the ultrasound energy 107.

It is within the scope of the present invention where the ultrasonic energy may be in a form of a continuous wave or pulsed wave.

According to one embodiment, the ultrasound energy is radiated from the implant surface 106. Yet more, the surface 106, is also exposed to acoustic waves, for instance as generated by the pulse wave when it flows through the blood vessel 104.

According to one embodiment, a smartphone application 102 may be utilized to communicate with the on-skin unit 100. In some embodiments, the application may run on external computer.

In some embodiments, the smartphone application 115, may communicate with a cloud-based program 103 for continuous monitoring and analysis.

Reference is now made to FIG. 1B illustrating substantially the same embodiment as illustrated in FIG. 1a with the addition of at least one transducer 116.

As detailed above, according to one embodiment, the implant comprises or is in communication with at least one sensor. Said sensor is adapted to monitor at least one physiological state of the patient. Once a change is detected the device is either self-activated to apply said energy (and thereby to induce NO release) or to notify the patient or any predefined care giver of the patient of said change and either the patient or the care giver activates the device.

According to one embodiment, the sensor is selected from a group consisting of a sensor implanted in said patient, a sensor being worn by said patient, a remote sensor outside the patient's body and any combination thereof. For example, the sensor can also be outside the body (e.g. a wearable) transmitting to the implant or it can be implanted elsewhere in the body. For example, a pacemaker that senses activity can also send a signal to our implant so both device work in concert and the heart rate increases while NO is released. Another example can be other sensors that are used in cardiology (e.g. heart failure implantable sensors like the BSC HeartLogic and the Abbot CardioMems).

According to other embodiments of the present invention, the implant is in communication with at least one on-skin controller; said controller is adapted to receive information associated with at least one physiological state of the patient. According to another embodiment, the controller is in communication with at least one sensor, adapted to sense information associated with at least one physiological state of the patient. Furthermore, according to said physiological state of the patient the controller can amend the treatment protocol of the applied ultrasound energy.

According to one embodiment, an ‘on-demand’ device/system will be provided. It is noted that the ‘on demand’ device will be activated only when needed (‘on-demand’ basis). According to this embodiment, the device will be integrated/in-communication with at least one sensor that monitors physiological state of the patient such that, upon change thereof, the device is activated or the treatment protocol of the device is amended.

According to this embodiment, the sensors of the ‘on-demand’ device could be selected from a group consisting of accelerometer, impedance measurement, Photoplethysmography, PPG sensor, pH sensor, Ultrasound sensor, hydrophone, temperature meter, manual activation, any sensor indicating a change in cardiac output, any sensor indicating blood pressure, any sensor indicating initiation of a dialysis session, any sensor associated with dialysis machine and any combination thereof.

Each of the sensors is adapted to monitor the physiological state of the patient and provide treatment accordingly. For example, accelerometer will be used to indicate if the position of the patient (e.g., when the patient is lying down, standing, walking etc.). Upon sense of a change (e.g., when the patient starts to walk) the device is activated. In other words, the device is activated ‘on demand’ (when the patient engages in physical activity, e.g., walking) to induce NO release (and thus, increase oxidation levels, tissue perfusion in the tissue and decrease any pain associated therewith).

Thus, it is another object of the present invention to provide the implantable device as defined above, wherein said sensor is selected so that indication of level of physical activity or changes thereof can be estimated for example initiation of walking or change in change in walking parameter such as speed, stride length, cadence or moving to standing from seating or laying down or moving from walking to running.

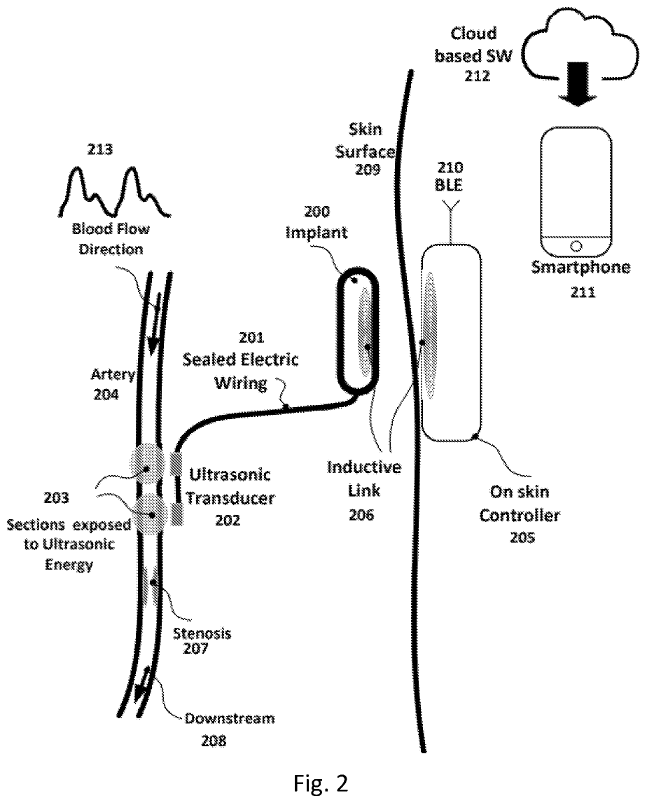

Reference is now made to FIG. 2, illustrating another embodiment of the present invention. According to this embodiment, the blood vessel 204, intended to be exposed to the ultrasound energy, is deeply located, or when there is no direct line of sight for ultrasound waves between an implant 200 to the blood vessel section to be treated 203.

According to this embodiment, the ultrasonic transducers 202 may be located in a sealed pig tail cable 201 that is coupled to the implant 200.

According to this embodiment, the implant 200 is controlled transcutaneously through an inductive link 206 (namely, at least one coil), by the on-skin unit 205.

The on-skin unit may communicate with a smartphone 211, using a wireless link such as BLE 210.

In another embodiment, said smartphone 211, may communicate with an external host program such as cloud-based software 212.

According to this embodiment, the end of the pig tail is located close to the blood vessel 204. Thus, one or more blood vessel sections 203 are exposed to the ultrasound generated by the ultrasonic transducers 202.

The ultrasonic transducers 202 may be piezoelectric transducers, electrostatic transducers, or any type of ultrasonic transducers capable of generating sufficient peak negative pressure to cause shear force in the blood vessel walls.

According to another embodiment, the ultrasonic transducers 202 may also be used as feedback sensors, to allow monitoring of the ultrasonic energy generated by the ultrasonic transducers.

In another embodiment, the ultrasonic transducers 202 may be employed to sense the blood pulse wave 213 when it flows in the artery 204 close to the pig tail 201.

In another embodiment, a dedicated acoustic sensor may be employed in the pig tail 201 for sensing.

In another embodiment, said acoustic sensor may sense the pulse wave 213 flowing through the artery 204 downstream 208

In another embodiment, a thermal sensor such as thermistor may be employed in the pig tail 201 for sensing.

In another embodiment, more than one acoustic sensor may be employed in the pig tail 201, in order to sense the pulse wave velocity in the blood vessel 204.

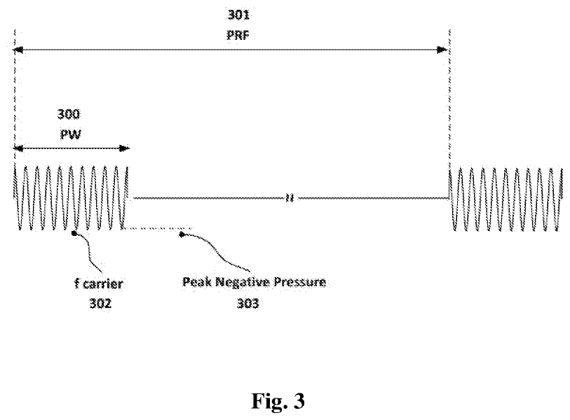

Reference is now made to FIG. 3 which illustrates a pulsed mode ultrasound energy.

The pulse wave duration PW 300 may be as short as 10 cycles. The pulse repetition frequency 301 may be such that the duty cycle may be as low as 10%. The ultrasonic energy pulse may have a peak negative pressure 303 as high as 5 MPa.

In another embodiment, the carrier frequency of the ultrasonic wave may be between 10 kHz-2 MHz.

In some embodiments, the ultrasonic carrier frequency 302 may be modulated.

In some embodiments, the pulse wave may be composed of more than one carrier frequency, in order to increase the shear force exerted on the blood vessel.

In some embodiments, the ultrasonic pulse amplitude may be amplitude modulated.

In some embodiments, the pule repetition rate, PRF, 301, and PW 300 length and amplitude 303 may be adjusted according to the patient's requirements.

In some embodiments, the PRF 301, PW 300 length and amplitude 303 may be adjusted automatically without the interventions of the patient.

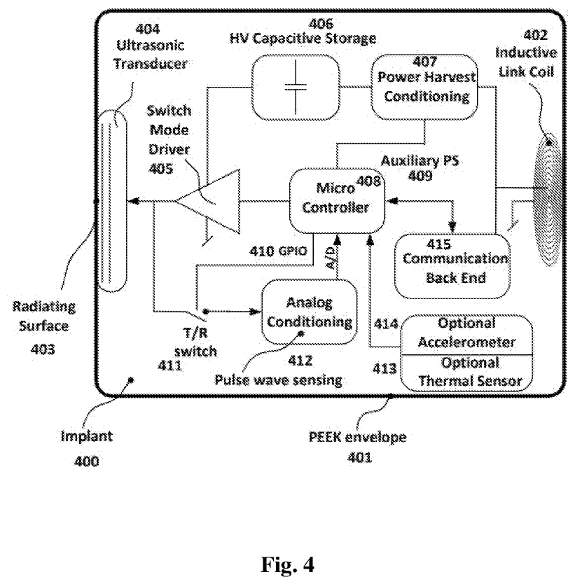

Reference is now made to FIG. 4 which illustrates a general overview of implant 400 realization.

According to this embodiment, the implant may have a non-metallic envelop, such as PEEK 401.

In some embodiments, the PEEK may be coated with Parylene.

The front surface 403 to which the ultrasonic transducers are attached internally, may serve as an acoustic matching layer, if its width has a thickness for instance of about a quarter of lambda, where lambda is the ultrasonic wavelength in the PEEK.

The front surface 403 is oriented toward the blood vessel to be treated, to allow the ultrasonic energy to hit the intended blood vessel. The radiating surface 403 is also used as a pressure wave receiving surface, such as to sense the pulse wave acoustic signal, or detect ultrasonic reflections.

According to one embodiment, the back surface of the implant employs a coil 402 as part of the inductive link with the on-skin unit. The energy harvested by the coil 402 may directly drive the ultrasonic transducers 404. In such case, rectification and filtering by the power harvest unit 407 is mainly required for the auxiliary power supply 409.

Alternatively, or additionally, the alternating electrical energy harvested by the coil 402, may be converted into direct voltage DC by the power harvesting unit 407. The DC voltage is stored in a capacitive storage 406. The energy collected at the capacitive storage 406, is used to drive an internal ultrasonic driver 405. The DC voltage is also used to power the implant circuitry by auxiliary power supply 409.

According to one embodiment, an analog conditioning stage 412 may be used to amplify and filter the pulse wave signal. To protect the analog circuitry from the higher voltage used to drive the ultrasonic transducer 404, a T/R switch 411 may be employed. The T/R switch may be controlled by a micro controller stage 408, for instance by a GPIO 410. The microcontroller samples the analog signal of the pulse wave, and may send it to the on-skin unit through the inductive channel.

The ultrasonic transducer 404 may be of PZT elements, preferably but not limited to hard Lead zirconate titanate PZT In order to reduce internal power loss.

In some embodiments, the piezoelectric may have a non-uniform lateral polarization such as Gaussian shaped polarization, in order to reduce side lobes in the ultrasonic energy.

In some embodiments, the ultrasonic transducer 404 may comprise an array of PZTs.

In some embodiments, the array of PZTs may be driven as a phased array.

According to one embodiment, the ultrasonic wave comprises more than one carrier frequency that may create higher shear force in the blood vessel walls. Therefore, in some embodiments, the array of PZTs 404, may be driven by more than one carrier frequency. For instance, part of the array is driven in one carrier frequency, and others by a second carrier frequency.

In some embodiments, an embedded microcontroller 408, may be employed. The microcontroller may control the transmit receive protection switch 411, collect the sensors information, control the ultrasonic driver 405, and communicates with the on-skin unit via communication block 415.

In some embodiments, an accelerometer sensor 414 may be employed to sense the movement of the patient.

In some embodiments, a thermal sensor 413 may be employed as a protective mean against over temperature of the implant circuitry and its envelope.

Blood pulse wave contains valuable information about the blood flow, such as blood velocity, pulse pressure level, augmentation index etc. Therefore, it may be beneficial to sense it for long term monitoring of the blood flow in the treated vessel and downstream tree.

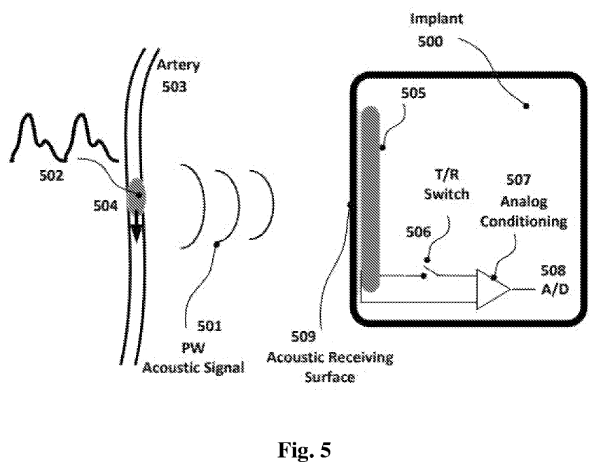

Reference in snow made to FIG. 5 which illustrates another embodiment of the present invention in which the pulse wave sensing configuration by the implant 500.

The blood pulse wave 502 flowing in the intended blood vessel (e.g., artery) 503 expands the blood vessel's walls radially 504 due to the elasticity of the blood vessel.

The momentary expansion and retraction of the blood vessel walls, generates an acoustic wave 501 that propagates through the tissue also toward the acoustic receiving surface 509 of the implant. Thus, the ultrasonic transducer 505 may serve also as an acoustic sensor.

Alternatively, a dedicated acoustic sensor may be employed, such as piezoelectric element made of a Piezoelectric lead-zirconate-titanate (PZT) or Polyvinylidene fluoride, or polyvinylidene difluoride (PVDF) membrane. To protect the analog conditioning circuitry 507 from the high drive voltage, a protection circuitry in the form of a T/R switch 506 may be employed. The amplified analog signal 508, may be sampled by the implant micro controller, and sent to the on-skin unit for further processing.

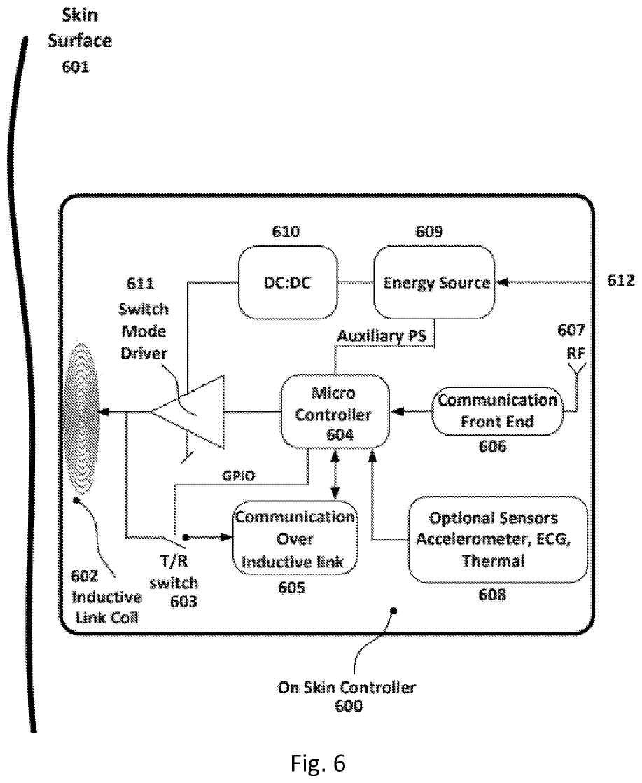

Reference in snow made to FIG. 6 which illustrates a general overview of a possible on skin controller 600 positioned on the skin 601. According to this embodiment, the controller employs a coil 602 that forms an inductive power and communication link with the implant coil.

A coil driver 611 is employed to drive the desired current to the coil. The driver 611 is powered by a power stage 610 which may be employed to boost the voltage of electrical energy source 609 to a desired level.

The energy source may be a non-rechargeable, or rechargeable battery which may be charged through port 612.

The on-skin controller 600 may employ a microcontroller 604 to facilitate communication channels with the implant though the inductive coil 602, and also incorporate a communication front end 606, to communicate with an external system through wireless link such as Bluetooth 607.

According to another embodiment, said microcontroller 604 may protect the receiving chain 605 by employing a transmit/receive electronic switch 603.

According to another embodiment, the on-skin unit may also employ several sensors 608, such as an accelerometer that enables the detection of the patient movements, a thermal sensor such as a thermistor for temperature protection, and an electrocardiogram (ECG) sensor. The ECG sensor facilitates the sensing of the propagation delay variations of the pulse wave in the treated artery compared to the ECG pulse.

The various sensors outputs, such as heart rate, pulse wave shape, pulse wave velocity, body core temperature, and the delay between the ECG and the pulse wave signal in the treated artery, may be analyzed locally by the on skin controller in order to adjust the ultrasound energy parameters, or sent upward for further processing and long-term data collection for instance by a cloud-based software.

Reference in snow made to FIG. 7 which illustrates a possible mechanism that facilitates data extraction through the inductive link. A bit stream data 700 modulates an electronic switch 701 such as Silicon switch to connect/disconnect an impedance 702 across the implant coil 703. The switched load impedance 702 draws switch current pulses that may be sensed by the on-skin coil.

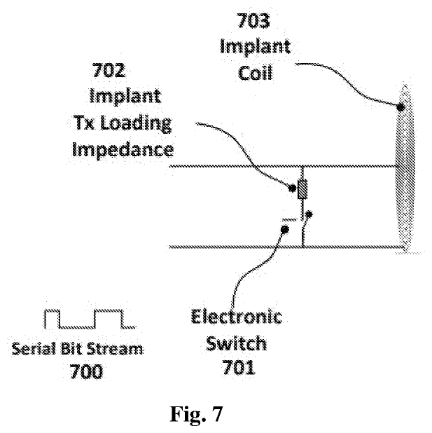

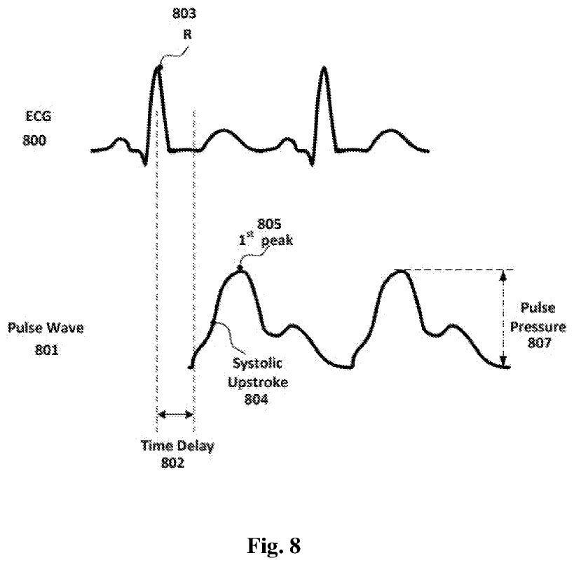

Reference in snow made to FIG. 8 which illustrates the time delay 802 between the ECG signal 800 and the pulse wave signal 801.

The time delay 802 may vary according to the blood flow resistance variations in the treated blood vessel and downstream. Typically, the time delay is measured between the R wave 803 of the ECG signal, to the start of the upstroke signal 804 of the pulse wave.

Furthermore, according to another embodiment, from the pulse wave signal, the pulse pressure 807 which is the difference between the systolic peak 805 and the diastolic end pressure is measured. The systolic upstroke time 804 is another possible parameter that depends on the blood flow downstream resistance. The overall information that may be extracted by the pulse wave and the ECG, may be used for automatic adjustment of the ultrasonic energy parameters generated by the implant or by the pig tail, and also for long term monitoring of the patient blood flow quality in the artery and downstream.

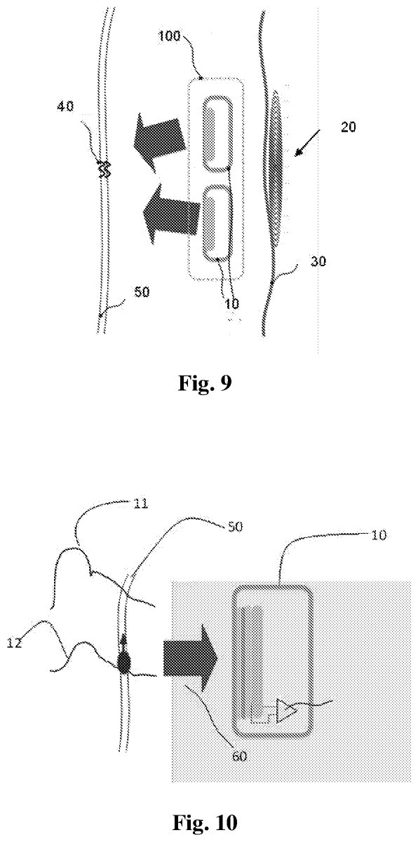

Reference is now made to FIG. 9 illustrating an array 100 of two implants, 10, being in communication with one external device (in this case, a coil), 20, located on the skin 30 of the patient. Upon activation of the transducers mechanical vibrations 40 are induced to the blood vessel 50. One skilled in the art would appreciate that while only 2 implants are illustrated in the figure, any no. of implants could be utilized.

According to this embodiment, utilizing a plurality of implant facilitates the use of small-sized implant.

Furthermore, by providing numerous implants, the position and orientation of each of the implants could be controlled. Yet more, control of the position and orientation of each of the implants provides focusing capability on the desired blood vessel can be achieved.

According to another embodiment of the present invention, as there are multiple implanted transducers, each could be operated in a different frequencies range (e.g., a first range of 20 kHz to about 10 MHz and a second range of 20 kHz to about 10 MHz). According to another embodiment of the present invention, all transducers operate in the same frequencies range.

The controller (external unit), which may be an electrical oscillator, may generate signals in the ultrasound frequency spectrum, e.g., as low as 20 kilohertz, or as high as 10 MHz.

According to other embodiments, the external controller is adapted to charge the implants. According to another embodiment, the charging is performed by at least one method selected from a group consisting of electromagnetic, ultrasonic, capacitive, induction and any combination thereof.

According to one embodiment, the controller comprising at least one coil.

Once the controller's signals are provided to the transducer, the transducer emits acoustic energy from its surface, as is well known to those skilled in the art.

The controller may control at least one selected from a group consisting of the amplitude, and therefore the intensity or power, of the acoustic wave transmitted by the transducer, the timing (start and period), possibly a refractory period (i.e. do not start a new session before at least X minutes passed from the last one), directionality of the signal (one transmitter can stimulate more than one blood vessels located slightly away from each other and the phased array can point the stimulation to a different one each time) and any combination thereof. In other embodiments, if the transducer includes more than one transducer elements, the controller may also control a phase component of the drive signals to respective transducer elements of the transducer device, e.g., to control a shape or size of a focal zone generated by the transducer elements and/or to move the focal zone to a desired location. For example, the controller may control the phase shift of the drive signals to adjust a focal distance (i.e., the distance from the face of the transducer to the center of the focal zone). In further embodiments, the controller can be configured to operate the transducer for a predetermined duration. Alternatively, or additionally, the controller can be configured to automatically turn off the transducer when a usage of the transducer exceeds said predetermined time.

It should be noted that the controller could be external to the patient or integrated within the implant. In such a way, when multiple implants are utilized and implanted, one implant (with the controller integrated therewithin) could control the remaining implants.

As disclosed above, according to one embodiment of the present invention, the controller can be in communication with at least one sensor selected from a group consisting of accelerometer, impedance measurement, Photoplethysmography, PPG sensor, pH sensor, Ultrasound sensor, hydrophone, temperature meter, body core temperature, heart pulse rate, Pulse wave properties, glucose sensor, manual activation, any sensor indicating a change in cardiac output, any sensor indicating blood pressure, any sensor indicating initiation of a dialysis session, any sensor associated with dialysis machine and any combination thereof. In such an embodiment the device is an ‘on-demand’ device. In other words, the ‘on demand’ device will be activated only when needed (‘on-demand basis). According to this embodiment, the sensor(s) s that monitors physiological state of the patient such that, upon change thereof, the controller signals the transducer to emit acoustic signal (or to amend one of the acoustic signal's parameters).

As specified, each of the sensors is adapted to monitor the physiological state of the patient and provide treatment accordingly. For example, accelerometer will be used to indicate if the position of the patient (e.g., when the patient is lied-down, stand, walk etc.). Upon sense of a change (e.g., when the patient starts to walk) the device is activated. In other words, the device is activated ‘on demand’ (when the patient engages in physical activity, e.g., walking) to induce NO release (and thus, increase oxidation levels/tissue perfusion in the tissue and decrease any pain associated therewith).

According to one embodiment of the present invention, the treated target tissue is one that has been affected by a peripheral arterial disease (and is located within the upper or lower limbs of the patient). In other embodiments, the target tissue can be associated with other diseases or medical conditions (such as pain due to exercising), and can be located at other parts of the patient.

Once the device is positioned in its predetermined location, the transducer (upon a signal from the controller) delivers ultrasound energy to the target tissue. The transducer may emit acoustic energy in a continuous manner, or alternatively, in pulses. In some embodiments, the controller may also control the phase, an operating frequency, temperature of the tissue (to ensure the same is not over heated) and/or an operating amplitude of the transducer.

According to another embodiment of the present invention, pulse wave characteristics and pulse wave velocity measurement can be measured to indicate artery dilation and increased blood perfusion. Reference is now made to FIG. 10 illustrating such acoustic sensing of pulse wave characteristics. As can be seen in the Fig. incident and reflected waves (11 and 12 respectively) results in vibrations 60 through the blood vessel 50 and can be sensed by either the external controller (or a micro controller embedded within the implant). Analysis of said waves can provide indication as for dilation and increased blood perfusion and for optimize orientation of the implant relative to the blood vessel. Thus, said analysis can facilitate alignment of the position of at least one of said plurality of implants relatively to said at least one vessel or tissue containing flowing blood.

According to another embodiment, the alignment is indicated (either to the patient or the care giver) by at least one indication means selected from a group consisting of audio means, visual means, tactile means and any combination thereof.

According to another embodiment, the analysis of said at least one pulse wave characteristic indicates if the desired physiologic effect (vessel dilation and increased blood perfusion) is indeed achieved.

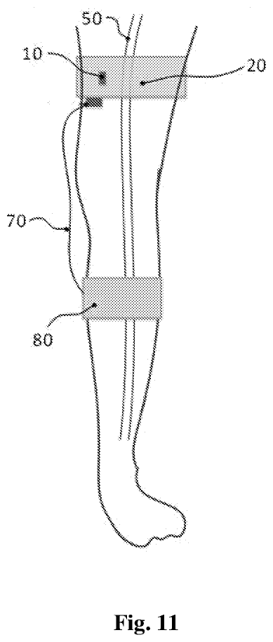

According to another embodiment of the present invention, a second momentary wearable controller is utilized to facilitate the pulse wave velocity flow measurement. Reference is now made to FIG. 11 illustrating such an embodiment.

According to this embodiment, implant 10 is in communication with the main external unit (the controller) 20. The main external unit 20 is wirely connected via electric wires 70 (however, it could be wireless connected) to at least one second wearable unit 80. Said second wearable unit 80 facilitates intermittent pulse wave flow sensing. According to one embodiment, the second wearable unit 80 may not be used continuously and only from time to time.

Alternatively, the second wearable unit 80 can be used as the battery holder for the main external unit (the controller) 20 thickness and weight.

According to one embodiment, an ‘on-demand’ device will be provided. It is noted that the ‘on demand’ device will be activated only when needed (‘on-demand basis). According to this embodiment, the device will be integrated with at least one sensor that monitors physiological state of the patient such that, upon change thereof, the device is activated or the treatment protocol of the device is amended.

According to this embodiment, the sensors of the ‘on-demand’ device could be selected from a group consisting of accelerometer, impedance measurement, Photoplethysmography, PPG sensor, pH sensor, Ultrasound sensor, hydrophone, temperature meter, body core temperature, heart pulse rate, Pulse wave properties, glucose sensor, manual activation, any sensor indicating a change in cardiac output, any sensor indicating blood pressure, any sensor indicating initiation of a dialysis session, any sensor associated with dialysis machine and any combination thereof.

Each of the sensors is adapted to monitor the physiological state of the patient and provide treatment accordingly. For example, accelerometer will be used to indicate if the position of the patient (e.g., when the patient is lied-down, stand, walk etc.). Upon sense of a change (e.g., when the patient starts to walk) the device is activated. In other words, the device is activated ‘on demand’ (when the patient engages in physical activity, e.g., walking) to induce NO release (and thus, increase oxidation levels, tissue perfusion in the tissue and decrease any pain associated therewith).

Thus, it is another object of the present invention where the sensor is selected to enable the indication of level of physical activity or changes thereof can be estimated for example initiation of walking or change in change in walking parameter such as speed, stride length, cadence or moving to standing from seating or laying down or moving from walking to running.

It is within the scope of this invention, wherein said sensor is selected from a group consisting of accelerometer, impedance measurement, Photoplethysmography, PPG sensor, pH sensor, Ultrasound sensor, hydrophone, temperature meter, body core temperature, heart pulse rate, Pulse wave properties, glucose sensor, manual activation, any sensor indicating a change in cardiac output, any sensor indicating blood pressure, any sensor indicating initiation of a dialysis session, any sensor associated with dialysis machine, any sensor indicating initiation of a dialysis session, any sensor associated with dialysis machine and any combination thereof.

Another example is the integration of impedance measurement sensor. According to this embodiment, vasodilation reduce local impedance around the vessel due to higher fluid volume compared to solid tissue. Thus, such impedance sensor will provide indication as for the NO/oxidation level in the tissue or blood vessel and therefrom, input could be retrieved as for the required activation of the device or any amendment needed to the treatment protocol of the device (increased US intensity, treatment duration time etc.).

According to another embodiment of the present invention, the implant will utilize a plurality of piezoelectric transducers. According another to embodiment the plurality of piezoelectric transducers will be arranged as a phased array. Such array will enable fine-tuning of the position of the device relatively to the treated blood vessel. According to this embodiment, each transducer will be activated/deactivated to enable aligning the position of the device relatively to the blood vessel to maximize the NO induction.

According to another embodiment, at least one of said piezoelectric transducers shaded by means of Gaussian apodization and material polarization.

According to another embodiment, the piezoelectric transducer is made of at least one material selected from a group consisting of lead zirconate titanate, lead magnesium niobate-lead titanate, Hard PZT, composite and any combination thereof.

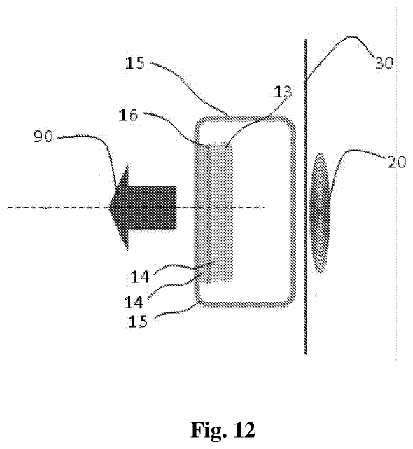

According to another embodiment, to reduce the overall implant thickness, multilayer acoustic matching is implemented. According to this embodiment, the implant encapsulation is a part of the acoustic matching layers. Reference is now made to FIG. 12 illustrating such an embodiment.

According to this embodiment, the implant is encapsulation in envelope made of an acoustic impedance matching; especially a layer of polyetheretherketone, PEEK, 15.

As seen in the Fig., the 1^{st} acoustic impedance matching layer 16 is provided distal-most and closest to the blood vessel. According to this embodiment, this 1^{st} acoustic impedance matching layer 16 is sandwiched between at least 2 layers of glue (e.g., penloc GTI) 14 and thereafter the transducer (e.g., piezoelectric element) 13 is positioned.

According to this embodiment, various piezoelectric materials can be employed, selected from Hard lead zirconate titanate, PZT, such as PZT8, lead magnesium niobate-lead titanate, PMN-PT, composite material and any combination thereof.

According to another embodiment, at least one of the implants comprises an ultrasound transducer (and, optionally, at least one ultrasound receivers), rechargeable battery and an array of sensors to measure physiological parameters (e.g. patient activity or local tissue perfusion).

According to another embodiment, an ‘on demand’ treatment is enabled. For example, the device can sense walking and applies therapy at the right timing to minimize pain.

It should be noted that local tissue perfusion and oxidation level can be measured by impedance, PPG, ultrasound echo and any combination thereof.

As described above, according to one embodiment of the present invention the implant is in communication with an external charging unit for wirelessly charging the implant.

According to some embodiments, the charging unit can be designed as a wearable unit worn by the patient. According to one embodiment, the charging unit will be worn once every predetermined time (e.g., once a day). According to another embodiment, the charging unit will be placed in a fixed location or integrated within the patient natural environment (e.g., furniture, e.g., under the bed's covers).

According to some embodiments, the charging unit will be performed by at least one method selected from a group consisting of electromagnetic, ultrasonic, capacitive, induction and any combination thereof. According to one embodiment, the charging unit (i.e., the external controller) comprising at least one coil.

According to other embodiments, the external controller will acoustically sense at least one pulse wave characteristic, reflected from at least one of the plurality of implants. It is within the scope of the present invention where the analysis of said at least one pulse wave characteristic facilitate alignment of the position of at least one of the plurality of implants relatively to the treated vessel or tissue containing flowing blood. It should also be appreciated that it is within the scope of the present invention where the analysis of said at least one pulse wave characteristic indicates if the desired physiologic effect has induced (e.g., artery dilation and increased blood perfusion).

According to one embodiment, such alignment is indicated (to the user of care giver) by audio means, visual means, tactile means and any combination thereof.

According to other embodiments, at least one of the implants will comprise at least one processor (or controller). According to one embodiment, such a processor can acoustically sense at least one pulse wave characteristic, reflected from at least one of the plurality of implants. It is within the scope of the present invention where the analysis of said at least one pulse wave characteristic facilitate alignment of the position of at least one of the plurality of implants relatively to the treated vessel or tissue containing flowing blood.

It should also be appreciated that it is within the scope of the present invention where the analysis of said at least one pulse wave characteristic indicates if the desired physiologic effect has induced (e.g., artery dilation and increased blood perfusion).

According to one embodiment, such alignment is indicated (to the user of care giver) by audio means, visual means, tactile means and any combination thereof.

According to other embodiments, at least one of the implants comprises at least one processor, adapted to control at least one parameter selected from a group consisting of a phase, an operating frequency, power, intensity, operating amplitude, temperature of the tissue, timing, duration of said source of energy and any combination thereof. According to such an embodiment, said implants are in communication with one another; such that, based on the parameters, the treatment protocol can be adjusted accordingly.

According to other embodiments, the processor is in communication with at least one sensor, adapted to sense information associated with at least one physiological state of the patient. The sensor could be any selected from a group consisting of a sensor implanted in said patient, a sensor being worn by said patient, a remote sensor outside the patient's body and any combination thereof.

According to other embodiments, the processor is adapted to collect data from said at least one sensor and perform at least one selected from a group consisting of: (a) monitor said data; (b) amend the treatment provided to said patient; (c) maintain the treatment provided to said patient as is; and any combination thereof.

According to another embodiment, the external unit, other than having the capabilities to charge the implant, will enable downloading the data collected by the sensors. According to some embodiments of the present invention, the data download will be enabled by means known in the art; e.g., similarly to RFID reading methods.

According to another embodiment of the present invention a data controller will be provided (e.g., in the form of a cellular application) enabling both clinicians and patient to control the device activity. The controller will be connected the data for monitoring and based thereupon amend/maintain the treatment protocol thereof.

According to some embodiments, the ultrasonic implant will be placed a few centimeters (e.g., 2-3 cm) from the intermittently or chronically ischemic or constricted (reduced perfusion) region or in an area where local enhancement of perfusion is desired either permanently or intermittently. Initially target arteries can be the lilac artery and the superficial femoral artery (SFA). After placing the implant, the treating physician will set the working parameters, activate it and verify the increase in perfusion at the ischemic or constricted (reduced perfusion) area.

Should the patient is found to be non-responsive (e.g. due to high degree of calcification in the artery not allowing dilation) then an additional implant can be considered. Furthermore, the patient's physiological response to local NO release can be tested using external ultrasound, verifying if significant portion is found to be non-responsive (e.g. due to high degree of calcification in the artery not allowing dilation).

Thus, it is noted that the patient physiological response to local NO release is tested using external ultrasound, verifying the device will be effective and estimate the effective NO dosage (which later will be used to set the working parameters of the device) and its response.

According to another embodiment of the present invention, compliance of the tissue or endothelial response to US application will be examined prior to utilizing the device. According to this embodiment, a secondary system (e.g., doppler ultrasound, ultrasound, MRI, CT) will be utilized. According to this embodiment, Doppler ultrasound device can be utilized for measuring a degree of perfusion, which provides a qualitative measure of the increase in blood flow resulting from the ultrasound treatment provided by the device.

According to another embodiment of the present invention, compliance of the tissue or endothelial response to US application will be examined prior to implantation. According to this embodiment, a secondary system (e.g., doppler ultrasound, ultrasound, MRI, CT) will be utilized. According to one embodiment, the secondary system is doppler ultrasound, ultrasound, MRI, CT etc. According to this embodiment, doppler ultrasound will applied at the required location (where the implant is thought to be implanted) and then at least one parameter selected from the group consisting of increase in blood flow (by analyzing the reflecting sound waves from red blood cells), NO levels, oxidation levels, tissue perfusion, verification of optimum position of said device and any combination thereof will be monitored to verify if there has been any improvement thereof. If, indeed, one of the parameters, e.g., the blood flow, has improved; that would indicate that the patient is responsive to the effect of the device and, hence, compatible for such use. Thus, a method of compatibility of patients to such treatment is provided. Alternatively, using such method could result in verification of optimum position of the implant as well as optimization of the treatment parameters and protocol to achieve best treatment.

Thus, according to this embodiment, at least one parameter of the device selected from a group consisting of intensity or power of the applied US energy, timing, duration thereof, frequency thereof, and any combination thereof is changed (while the other parameters are maintained constant) and the effect thereof on the treated tissue or blood vessel is examined via the secondary system (the doppler ultrasound, ultrasound, MRI, CT). In this way, patient's tailor-made & optimized treatment protocol can be established.

According to one embodiment, the implant according to the present invention comprises at least one transducer. According to other embodiments, the transducer is either piezoelectric transducer which generates ultrasound energy, at least one passive ferromagnetic element and any combination thereof.

According to other embodiments, the transducer may be provided on a platform capable of supporting thereof. The platform may be substantially rigid, semi-rigid, or substantially flexible, and can be made from a variety of materials, such as plastics, polymers, metals, and alloys. Electrodes and conducting wires may also be provided in a known manner for coupling the transducer to the control.

According to one embodiment of the present invention, the transducer includes one or more transducer elements. Each of the transducer element(s) may be a one-piece piezoceramic part, or alternatively, be composed of a mosaic arrangement of a plurality of small piezoceramic elements (e.g., phased array). The piezoceramic parts or the piezoceramic elements may have a variety of geometric shapes, such as hexagons, triangles, squares, and the like. The material used to construct the transducer element(s) could be a composite material, a piezoceramic, or any other material that could transform electrical signal into acoustic wave. The transducer element(s) are coupled to the controller for generating and/or controlling the acoustic energy emitted by the transducer element(s).

According to other embodiments, the piezoelectric is made of at least one material selected from a group consisting of lead zirconate titanate, lead magnesium niobate-lead titanate, Hard PZT, composite and any combination thereof.

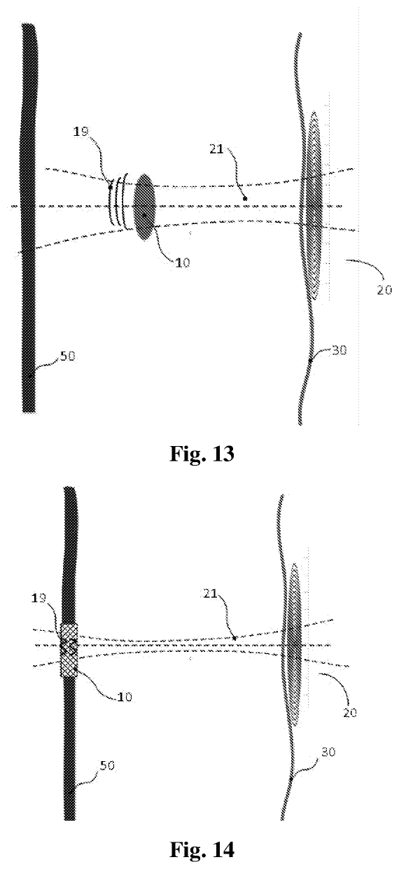

According to another embodiment of the present invention, a passive implant is provided, according to which an electro-magnetic acoustic transducer, EMAT, is provided, to induce ultrasonic vibrations. Reference is now made to FIG. 13 illustrating such an embodiment.

As seen in the Fig., implant 10 is made of an EMAT (made of ferromagnetic body) to induce ultrasonic vibration 19 to the blood vessel 50.

The external control (illustrated as a coil in the FIG. 20 generates alternating electro-magnetic flux 21 that results in mechanical vibrations from the EMAT onto the blood vessel 50.

According to another embodiment of the present invention, the NO induction is provided by utilizing an already implanted stent. Reference is now made to FIG. 14 illustrating such an embodiment. As seen in the Fig., ferromagnetic stent 10 which was previously implanted in the patient is utilized to induce ultrasonic vibrations. As previously described, the external unit (the external controller (shown in the Fig. as a coil) 20 generates alternating flux that induce current in the ferromagnetic surface of stent 10. This, in turn, results in mechanical vibrations onto the blood vessel 50.

As indicated above, the controller may control at least one selected from a group consisting of the amplitude, and therefore the intensity or power, of the acoustic wave transmitted by the transducer, the timing (start and period), possibly a refractory period (i.e. do not start a new session before at least X minutes passed from the last one), directionality of the signal (one transmitter can stimulate more than one blood vessels located slightly away from each other and the phased array can point the stimulation to a different one each time) and any combination thereof. In other embodiments, if the transducer includes more than one transducer elements, the controller may also control a phase component of the drive signals to respective transducer elements of the transducer device, e.g., to control a shape or size of a focal zone generated by the transducer elements and/or to move the focal zone to a desired location. For example, the controller may control the phase shift of the drive signals to adjust a focal distance (i.e., the distance from the face of the transducer to the center of the focal zone). In further embodiments, the controller can be configured to operate the transducer for a predetermined duration. Alternatively, or additionally, the controller can be configured to automatically turn off the transducer when a usage of the transducer exceeds said predetermined time.

It should be noted that the controller could be external to the patient or integrated within the implant. In such a way, when multiple implants are utilized and implanted, one implant (with the controller integrated therewithin) could control the remaining implants.

As disclosed above, according to one embodiment of the present invention, the controller can be in communication with at least one sensor selected from a group consisting of accelerometer, impedance measurement, Photoplethysmography, PPG sensor, pH sensor, Ultrasound sensor, hydrophone, temperature meter, body core temperature, heart pulse rate, Pulse wave properties, glucose sensor, manual activation, any sensor indicating a change in cardiac output, any sensor indicating blood pressure, any sensor indicating initiation of a dialysis session, any sensor associated with dialysis machine and any combination thereof. In such an embodiment the implant device is an ‘on-demand’ device. In other words, the ‘on demand’ device will be activated only when needed (‘on-demand basis). According to this embodiment, the sensor(s) s that monitors physiological state of the patient such that, upon change thereof, the controller signals the transducer to emit acoustic signal (or to amend one of the acoustic signal's parameters).

As specified, each of the sensors is adapted to monitor the physiological state of the patient and provide treatment accordingly. For example, accelerometer will be used to indicate if the position of the patient (e.g., when the patient is lied-down, stand, walk etc.). Upon sense of a change (e.g., when the patient starts to walk) the device is activated. In other words, the device is activated ‘on demand’ (when the patient engages in physical activity, e.g., walking) to induce NO release (and thus, increase oxidation levels/tissue perfusion in the tissue and decrease any pain associated therewith).

According to one embodiment of the present invention, the treated target tissue is one that has been affected by a peripheral arterial disease (and is located within the upper or lower limbs of the patient). In other embodiments, the target tissue can be associated with other diseases or medical conditions (such as pain due to exercising), and can be located at other parts of the patient.

Once the device is positioned in its predetermined location, the transducer (upon a signal from the controller) delivers ultrasound energy to the target tissue. The transducer may emit acoustic energy in a continuous manner, or alternatively, in pulses. In some embodiments, the controller may also control the phase, an operating frequency, temperature of the tissue (to ensure the same is not over heated) and/or an operating amplitude of the transducer.

As noted above, the delivered acoustic energy by the transducer is at least partially absorbed by the tissue, and causes mechanical stimulation of endothelial cells by compression and wall shear stress in blood vessels, thereby stimulating production of endothelial nitric oxide syntheses (eNOs). The heightened level of nitric oxide is believed to have a number of effects on the tissue, including inhibition of leukocyte and platelet adhesion, control of vascular tone and maintenance of a thromboresistant interface between the bloodstream and the vessel wall, increase in capillary circumference (vasodilation), and/or increase in blood flow (perfusion), treat pulmonary HP. Such effect(s) in turn helps relieve pain at the tissue, and allows the patient to rehabilitate through exercise.

According to another embodiment of the present invention, a secondary system is utilized. According to this embodiment, the secondary system is at least one selected from doppler ultrasound, ultrasound, MRI, CT and any combination thereof. According to this embodiment, doppler ultrasound will applied at the required location and then at least one parameter selected from the group consisting of blood flow (by analyzing the reflecting sound waves from red blood cells), NO levels, oxidation levels, tissue perfusion, verification of optimum position of said device and any combination thereof will be monitored to verify if there has been any improvement thereof. If indeed one of the parameters, e.g., the blood flow, has improved; that would indicate that the patient is responsive to the effect of the device and, hence, compatible for such use. Thus, a method of compatibility of patients to such treatment is provided.

According to another embodiment of the present invention, the secondary system (e.g., doppler ultrasound, ultrasound, MRI, CT) will be utilized to optimized treatment protocol. According to this embodiment, doppler ultrasound (or ultrasound, MRI, CT) will applied at the required location and then at least one parameter selected from the group consisting of blood flow (by analyzing the reflecting sound waves from red blood cells), NO levels, oxidation levels, tissue perfusion, verification of optimum position of said device and any combination thereof will be monitored to establish optimized treatment parameters. Thus, according to this embodiment, at least one parameter of the implant device selected from a group consisting of intensity or power of the applied US energy, timing, temperature of the treated tissue (to make sure the same is not overheated), duration thereof, frequency thereof, and any combination thereof is changed (while the other parameters are maintained constant) and the effect thereof on the treated tissue or blood vessel is examined via the secondary system (the doppler ultrasound or ultrasound, MRI, CT). In this way, patient's tailor-made & optimized treatment protocol can be established.

It should be noted that according to this embodiment, during a treatment session, the energy intensity or dosage delivered by the transducer at the tissue is kept below a prescribed threshold (e.g., by using appropriate driving scheme and/or by selecting appropriate operation parameters, such as an operating frequency, an operating amplitude, etc.), thereby protecting the tissue from being injured by the acoustic energy.

As noted above, in any of the embodiments described herein, the implant can further include one or more additional ultrasound transducer(s). The transducers can be positioned in a side-by-side configuration to form a line. For example, in some embodiments, the system includes several transducers, each of which operates in different delivered acoustic wave. It is noted that providing a plurality of transducers allows treatment of multiple target regions simultaneously or treatment at different treatment protocol (e.g., each transducer operates in different power, intensity, timing, duration etc.).

In some cases, the controller can be configured to control the transducers such that acoustic waves emitted by the respective transducers interact in a desired manner. For example, in some embodiments, a relative phase between transducers may be varied. In one implementation, adjacent transducers are alternately driven in phase and out of phase. Because the acoustic fields from adjacent transducers may overlap and because of resonance, the intensity distribution within a patient's body may form a series of interference maxima and mina. By altering the phase relation between the transducers, the locations of these pecks and nulls may be reversed, thereby providing overall uniform (or substantially uniform) insonification at target tissue or blood vessel. In other embodiments, the operating frequency of one or more transducers may be varied to move an interference pattern of the acoustic field.

In other embodiments, the transducer can be moved relative to the patient. In such embodiment, the position of at least one of the transducers can be optimized relatively to the desired treated tissue or blood vessel.

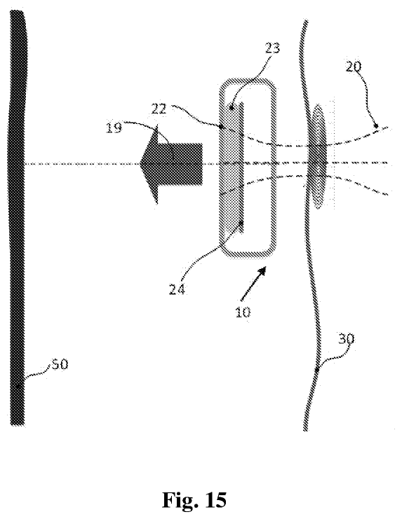

In other embodiments, at least one of the implants are adapted to apply ultrasonic energy in at least two substantially different frequencies of the carrier signal. Reference is now made to FIG. 15 illustrating such an embodiment. In such an embodiment, the implant 10 comprises at least one piezoelectric transducer 23 adapted to generate said ultrasonic energy in one of said two substantially different frequencies of the carrier signal; and at least one electro-magnetic acoustic transducer, EMAT, 24 mechanically coupled to said at least one piezoelectric transducer 24, adapted to generate said ultrasonic energy in a second, different, frequencies range. As described above, subject to activation of the external controller 20, mechanical vibrations (acoustic wave) 19 are induced upon the blood vessel 50.

In one embodiment, the first range is selected from about 20 kHz to about 10 MHz and the second range is selected from about 20 kHz to about 10 MHz.

In other embodiments, the at least one electro-magnetic acoustic transducer is at least one ferromagnetic sheet.

In other embodiments, the implants are encapsulated in at least one layer of polyetheretherketone, PEEK, 22.

According to another embodiment of the present invention, multiple implants are utilized. One of which is very thin, comprises at least one coil and positioned close to the skin; while at least one second implant comprises the transducer (the piezoelectric element) positioned close to the blood vessel. Both the implants are wirely coupled to each other. According to another embodiment, the thin implant (that comprises at least one coil and positioned close to the skin) is wirely connected to a plurality of implants, each of which being positioned at a different position and orientation with respect to the blood vessel. Such an embodiment provides multiple point of treatment along the blood vessel (resulting in a large range of influence) and, as discussed above, may analysis the pulse wave characteristics to optimize treatment parameters and positioning (e.g., alignment) of at least one of the implant relative to the blood vessel. It should be noted that the implant device could be used for the treatment of:
CLI/PAD—Improved healing for post-revascularization patients. In such application the implant will be activated following revascularization either constantly or intermittently.
Improved PAD patients' wound healing, including saving legs from amputation.
Local and chronic therapy for pulmonary artery hypertension.
Treating Severe Asthma patients (severe patients which are not responding to the standard Asthma inhalers) by affecting the bronchial arteries. It should be noted that in such application, any parameters associated with breathing or vocal which can provide indication of an asthma attach can trigger the activation of the implant device to alleviate the bronchial spasm.
Improve blood flow to the brain during stroke by generating NO in the carotid artery (using an external ultrasound unit).
“Local Viagra”—increase blood flow to the penis to maintain an erection without the systemic side effects of sildenafil (the Viagra pill).
Improved bioavailability of medication—it should be noted that for this application, the time of medication administration (oral, IV or pump activation) can be synchronized to the activation of the ultrasound implant to enhance local absorption of the medication at the target organ and the location of the implant would then be in the vicinity of arteries feeding said target organ. For example, chemotherapy flow into a solid tumor could be enhanced during chemotherapeutic sessions.

Although certain embodiments have been illustrated and described herein, it will be appreciated by those of ordinary skill in the art that a wide variety of alternative or equivalent embodiments or implementations, calculated to achieve the same or similar purposes, may be substituted for the embodiments illustrated and described herein without departing from the scope of the present invention. Those of skill in the art will readily appreciate that embodiments in accordance with the present invention may be implemented in a very wide variety of ways. This application is intended to cover any and all adaptations and/or variations of the embodiments discussed herein.

The terms and expressions which have been employed in the foregoing specification are used therein as terms of description and not of limitation, and there is no intention, in the use of such terms and expressions, to exclude equivalents of the features shown and/or described or portions thereof, it being recognized that the scope of the invention is defined and limited only by the claims that follow.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range.

1-80. (canceled)

81. A device adapted to be implanted adjacent to at least one vessel or tissue containing flowing blood, the device comprising:
at least one ultrasonic transducer configured to emit ultrasonic energy toward the at least one vessel or tissue, so as to cause a physiologic effect in the at least one vessel or tissue;
wherein the device is configured to be in communication with at least one on-skin remote controller positioned externally to the patient and configured to control the ultrasonic energy; and
wherein the device is configured to be in communication with at least one sensor configured to monitor at least one physiological state of the patient.

82. The device according to claim 81, wherein the at least one ultrasonic transducer comprises an array of ultrasonic transducers.

83. The device according to claim 82, wherein the array of ultrasonic transducers is configured to facilitate alignment of the ultrasonic energy relative to the at least one vessel or tissue.

84. The device according to claim 81, wherein the ultrasonic energy is provided in at least two different frequencies of a carrier signal.

85. The device according to claim 81, wherein the emitting of the ultrasonic energy is synchronized to a pulse wave signal.

86. The device according to claim 81, wherein the at least one ultrasonic transducer is configured to function as an acoustic sensor and sense one or more acoustic waves generated by the at least one vessel or tissue.

87. The device according to claim 81, comprising at least one coil configured to form an inductive link between the device and the at least one on-skin remote controller.

88. The device according to claim 87, comprising:
at least one implant comprising the at least one coil; and
at least one pig tail comprising the at least one ultrasonic transducer;
wherein the at least one pig tail is configured to be wired or wirelessly connected to the at least one implant.

89. The device according to claim 81, wherein the monitored physiological state comprises one or more of: position of the patient, physical activity of the patient, nitric oxide levels in the at least one vessel or tissue, tissue perfusion, medical treatment provided to the patient, and any combination thereof.

90. The device according to claim 81, wherein the physiologic effect comprises one or more of: vasodilation, increase in local ATP, enhanced ATP release, increase in local nitric oxide, prolonged local nitric oxide effects, enhanced nitric oxide release from vascular endothelium, enhanced nitric oxide release from red blood cells, alteration in erythrocytes function, modification in oxygen release from hemoglobin, increase in blood temperature, modification in pH of blood, modulation in immune response of blood leucocytes, modulation of coagulation and/or thrombocyte function, modification in function of heme catalyst enzymes in the blood, improved bioavailability of medication, improved efficiency of a hemodialysis session, artery dilation, increased blood perfusion, and any combination thereof.

91. The device according to claim 81, wherein upon change of the at least one physiological state one or more of the following is performed: (a) the device is activated and ultrasonic energy is emitted, such that an on-demand treatment is provided; (b) at least one parameter of the ultrasonic energy is amended, such that an as-needed treatment is provided; (c) a notification is generated.

92. The device according to claim 81, wherein the device is configured to be automatically activated upon change of the at least one physiological state.

93. The device according to claim 81, wherein the device comprises one or more of the at least one sensor.

94. A system comprising:
at least one implantable device adapted to be implanted adjacent to at least one vessel or tissue containing flowing blood, the at least one implantable device comprising at least one ultrasonic transducer configured to emit ultrasonic energy toward the at least one vessel or tissue, so as to cause a physiologic effect in the at least one vessel or tissue;
at least one on-skin remote controller configured to be positioned externally to the patient and configured to control the ultrasonic energy; and
at least one sensor configured to monitor at least one physiological state of the patient;
wherein the at least one implantable device is configured to be in communication with the at least one on-skin remote controller and with the at least one sensor.

95. The system according to claim 94, wherein the at least one on-skin remote controller is configured to collect data from the at least one sensor and perform at least one of: monitor the data, adjust a treatment provided to the patient, maintain the treatment provided to the patient as is, and any combination thereof.

96. The system according to claim 95, wherein the data comprises one or more of: shape of a pulse wave signal, velocity of a pulse wave signal and a time delay between an ECG signal and the pulse wave signal.

97. The system according to claim 94, wherein the at least one on-skin remote controller is configured to control at least one parameter of the ultrasonic energy, the at least one parameter being one or more of: phase, frequency, power, intensity, amplitude, timing, duration, and any combination thereof.

98. The system according to claim 94, wherein the at least one sensor is configured to be one or more of: implanted in the patient, integrated within the implantable device, worn by the patient, integrated within the at least one on-skin remote controller, and any combination thereof.

99. The system according to claim 94, wherein the at least one sensor comprises one or more of: an accelerometer, a temperature sensor, a pulse wave sensor and an electrocardiogram (ECG) sensor.

100. The system according to claim 94, wherein one or more of the at least one implantable device and one or more of the at least one on-skin remote controller comprise at least one coil configured to form an inductive link between the at least one implantable device and the at least one on-skin remote controller.

101. A method of treating a patient using an implantable device adapted to be implanted adjacent to at least one vessel or tissue containing flowing blood, the method comprising:
using at least one ultrasonic transducer of the implantable device, emitting ultrasonic energy toward the at least one vessel or tissue, so as to cause a physiologic effect in the at least one vessel or tissue;
using at least one sensor, monitoring at least one physiological state of the patient; and
using at least one on-skin remote controller, controlling at least one of an operation of the implantable device and one or more parameters of the emitted ultrasonic energy, based on data collected by the at least one sensor.
